(19) United States
(12) Patent Application Publication
(10) Pub. No.: US 2024/0307536 A1
(43) Pub. Date: Sep. 19, 2024

(54) IMPROVED ADOPTIVE CELL TRANSFER THERAPY FOR CANCER

(71) Applicant: Hadasit Medical Research Services and Development Ltd., Jerusalem (IL)

(72) Inventors: Amnon PELED, Tel-Aviv (IL); Inbal MISHALIAN, Mevasseret Zion (IL)

(73) Assignee: Hadasit Medical Research Services and Development Ltd., Jerusalem (IL)

(21) Appl. No.: 18/271,883

(22) PCT Filed: Jan. 11, 2022
(86) PCT No.: PCT/IL2022/050038

§ 371 Date: Jul. 12, 2023

Related U.S. Application Data
(60) Provisional application No. 63/136,217, filed on Jan. 12, 2021.

(51) Int. Cl. A61K 39/00; A61P 35/02; C07K 14/54; C07K 14/55; C07K 14/705; C07K 14/715; C07K 16/28; C12N 5/0783; C12N 9/64

(52) U.S. Cl. CPC A61K 39/4611; A61K 39/4631; A61K 39/4635; A61K 39/464402; A61K 39/464412; A61P 35/02; C07K 14/5443; C07K 14/55; C07K 14/7056; C07K 14/7158; C07K 16/2803; C07K 16/2866; C12N 5/0638; C12N 9/6467; C12Y 304/21079; A61K 2239/26; A61K 2239/48; C07K 2317/622; C07K 2317/73; C12N 2501/2302; C12N 2501/515; C12N 2510/00

(57) ABSTRACT

The present invention is directed to the field of immunotherapy. Specifically, the invention provides improved cell compositions and methods for adoptive cell therapy, useful in the treatment of cancer. More specifically, embodiments of the invention employ the use of cell compositions comprising a high proportion of activated cytotoxic CD8⁺ cells and in particular CD8⁺NKG2D⁺granzyme-B⁺ cells characterized by enhanced cytotoxicity, to processes for their preparation from peripheral blood mononuclear cells (PBMC), and to their use in cancer management.

### FIELD OF THE INVENTION

The present invention is directed to the field of immunotherapy. Specifically, the invention provides improved cell compositions and methods for adoptive cell therapy, useful in the treatment of cancer.

### BACKGROUND OF THE INVENTION

Adoptive Cell Transfer (ACT, also referred to as adoptive cell therapy) is one of the most promising strategies in cancer immunotherapy, with a proven efficacy against a variety of cancer types, including melanoma, cervical cancer, lymphoma, leukemia, bile duct cancer, and neuroblastoma.

ACT is a cell therapy that attempts to enhance the anti-tumor activity of immune cells. It is based on immune cells extracted from a subject, which are typically processed ex-vivo and extensively expanded, and then transferred back to the patient (autologous therapy) or to another individual (allogenic therapy). Various immune cells including T lymphocytes (T cells), natural killer (NK) cells, dendritic cells, and stem cells can be used in ACT.

Tumor-infiltrating lymphocytes (TIL) are tumor-specific T cells that are isolated from a tumor biopsy of a patient and expanded ex vivo to produce ACT compositions. To this end, TIL are encouraged to multiply ex vivo using high concentrations of IL-2, anti-CD3 antibodies and irradiated allo-reactive feeder cells, wherein IL-2 (but not anti-CD3) is supplemented on day 5. The cells resulting after 8-15 days of expansion and then are transferred back into the patient along with supportive IL-2 administration. However, generation of TIL compositions for ACT is technically challenging, and produces undesired variability due to the use of IL-2 and feeder cells. Further, the approach is limited by the availability of tumor samples and by the low number of initial TIL that are typically obtained from each sample.

Another source that can be used for producing ACT compositions is peripheral blood mononuclear cells (PBMC), from which lymphocyte populations may be isolated, expanded and/or otherwise manipulated by various procedures, to enhance their efficacy. Unlike T cells, which exert a T cell receptor (TCR)-mediated, Major Histocompatibility Complex (MHC)-II-dependent cytotoxicity, and thus need to be autologous or histocompatible with the treated subject, other lymphocyte populations such as NK cells (characterized by the expression of CD56 or CD16 and the absence of the TCR-CD3 complex) and NK-T cells act in a non-MHC II-restricted fashion. However, these populations are typically characterized by limited efficacy, and require large doses in order to show clinical effects.

For example, lymphokine-activated killer (LAK) cells, consisted of various lymphocyte types such as natural killer (NK) cell precursors and T lymphocytes, are produced by culturing of PBMC in the presence of cytokines, typically IL-2. These heterogeneous cell compositions exhibit HLA-independent lysis of tumor cells in-vitro following incubation with IL-2. Although LAK cells can be highly effective in mediating tumor regression in certain individuals, this treatment approach may be difficult to perform since very large numbers of cells are required to mediate cancer regression. In addition, the high doses of IL-2 required mediate toxic side effects, the most common of which is a capillary permeability leak syndrome that results in major fluid retention.

Cytokine-induced killer (CIK) cells are non-MHC restricted, cytotoxic antitumoral cells expanded in vitro from circulating precursors. CIK cells share characteristics of both T and NK cells. The expansion protocol for the generation of CIK cells, starting from PBMC, lymphocytoapheresis or cord blood requires the sequential addition of 1000 U/mL human recombinant human interferon-gamma (rIFN-7) on day 0 followed by 50 ng/mL monoclonal antibody against CD3 (OKT3) and 500 IU/mL IL-2 on day +1. IL-2 and fresh complete X-VIVO medium (but not the anti-CD3 antibody) are added every five days for 14-21 days. At the end of this process, the generation of two main cell populations has been reported: one which comprise CD3, CD8, NKG2D and CD56 positive cells (usually referred to as CIK), and another population which includes CD3, CD8 and NKG2D positive but CD56 negative cells, reported to lack cytotoxic activity (Introna et al, Int. J. Mol. Sci. 2018, 19, 358).

Despite the relative safety profile of CIK, their use in clinical practice has resulted in extreme heterogeneity with respect to efficacy. Results of 11 clinical trials investigating the anti-tumoral effect of CIK cells on 426 treated patients showed that among 384 (90.1%) reported response, only 24 (5.6%) had a complete response, 27 (6.3%) had a partial response (PR), and 40 (9.4%) had a minor response. On the other hand, 161 (37.8%) patients had stable disease and 129 (30.3%) progressed, indicating an overall modest efficacy of CIK cell therapy (Introna et al, ibid).

Protocols for isolation and/or ex vivo expansion of various cell compositions for ACT have been disclosed. Typically, such protocols often include initial stimulation in the presence of cytokines and/or other stimuli such as TCR/CD3 activators, and expansion or propagation in the presence of distinct combinations of such cytokines and activators. In addition, such protocols may include additional steps of isolating or enriching for desired cell populations e.g. by apheresis or by flow cytometry or bead-based methods targeting the corresponding surface markers, either prior to or following expansion.

For example, US2020138861 discloses an adoptive transfer procedure for treating inflammatory bowel disease comprising isolating CD3 negative and CD56 positive NK cells from peripheral blood of a donor. U.S. Ser. No. 10/149,863 discloses a preparation method of isolated mammal NKT-like cells, comprising in vitro culturing isolated mononuclear cells, and adding culture cytokine(s) that stimulate the proliferation and activation of T cell, and sorting the NKT-like cells by using a cell sorting technique with NKT-like cell surface markers.

US2012258085 relates to a method of obtaining expanded and activated NK cells with the phenotype CD3⁻CD56⁺ and NK-like T cells with the phenotype CD3⁺CD56⁺, comprising providing a cell sample of peripheral blood from a tumor bearing subject, incubating and expanding the cells until at least 35% of the expanded cell population comprises activated NK cells and NK-like T cells; and harvesting said expanded cells. In particular, US2012258085 discloses a method whereby PBMC are initially cultured in the presence of monoclonal anti-CD3 antibody (OKT-3, 10 ng/ml) and 500 U/ml IL-2, wherein after five days, the cultures were replenished with fresh medium with 5% human serum and IL-2 but without OKT-3, every 2-3 days until the end of the culture.

Due to the limited efficacy of many ACT compositions, the use of genetically modified cells has been suggested. These include for example T-cells propagated from PBMC (or other lymphocyte populations such as NK cells) expressing cloned recombinant TCR αβ chains recognizing epitopes from shared tumor associated antigens (TAAs), or expressing chimeric antigen receptors (CAR) composed of immunoglobulin variable regions recognizing tumor antigens fused to signaling domains of the TCR (chain and costimulatory molecules, such as CD28 and CD137/4-1BB. For example, US2020345779 and US2020223920 disclose CAR polypeptides that can be expressed in various cell populations including αβT cells, γδT cells, NK cells, NKT cells, B cells, innate lymphoid cells (ILC), CIK cells, cytotoxic T lymphocytes (CTL), LAK cells, and regulatory T cells.

Despite the developments in cancer management in the recent years, there remains a need for additional means, improved methods and cell compositions that can be used in cancer immunotherapy. In particular, non-responsiveness to treatment, either primary or secondary, is highly prevalent among cancer patients, and many existing treatments are characterized by considerable adverse effects. The development of therapeutic modalities providing enhanced efficacy and/or safety would thus be highly advantageous. In addition, it would be beneficial to obtain cell compositions for ACT exhibiting improved survival and/or enhanced tumor engagement or retention.

### SUMMARY OF THE INVENTION

The present invention is directed to the field of immunotherapy. Specifically, the invention provides improved cell compositions and methods for adoptive cell therapy (ACT), useful in the treatment of cancer. More specifically, embodiments of the invention employ the use of cell compositions, herein designated super-activated killer cells (SAK), comprising a high proportion of activated CD8⁺ cells and in particular CD8⁺NKG2D⁺ granzyme-B⁺ cells, and characterized by enhanced cytotoxicity. The invention further relates to processes for the generation of SAK compositions from peripheral blood mononuclear cells (PBMC), and to their use in cancer management. According to advantageous embodiments, the invention further relates to genetically modified SAK compositions, exhibiting improved survival and/or enhanced tumor engagement and retention.

The invention is based, in part, on the development of SAK compositions, capable of exerting a highly potent anti-tumor cytotoxic activity against a variety of tumor cells. Surprisingly, SAK cells, produced from PBMC by a unique culturing process as disclosed herein, were significantly more effective at eradicating tumor cells than other PBMC-derived effector cell compositions, including cytokine-induced killer cells (CIK) and interleukin-2 (IL-2)-expanded T cell compositions, produced according to hitherto known ACT protocols. Further, the generated SAK cells were found to be exceptionally effective in inhibiting tumor development in vivo, in a human lymphoma (Raji) engraftment murine model.

As disclosed herein, the generated SAK populations were found to be characterized by high NKG2D levels, detected on the surface of the entire CD8⁺ cell fraction, and to be particularly effective against tumors expressing NKG2D ligands. As further disclosed herein, while the SAK populations were characterized by high expression levels of FasL, Granzyme B and perforin, their anti-tumor cytotoxic activity was found to be mediated mainly by Granzyme B and perforin, and was mostly insensitive to the use of anti-FasL neutralizing antibodies, despite the requirement for cell-cell contact. This is in contradistinction from hitherto known cell populations for ACT, in which the cytotoxic activity was reportedly substantially FAS-mediated. In addition, the cells were demonstrated to display an advantageous profile of cell-surface receptors, including abundant expression of receptors known to have a positive effect on the survival, expansion and/or activity of effector immune cells, and minimized expression of inhibitory checkpoint molecules known to be associated with exhaustion and/or activation-induced cell death. As further demonstrated herein, SAK cells were found to possess both MHC-dependent and non-MHC dependent cytotoxic activities against various tumor cells. Further, adoptive transfer of SAK cells provided for improved safety, manifested as a markedly reduced risk of invoking graft-versus-host disease (GVHD), and to retain a high level of activity even following cryopreservation. The invention is also based, in part, on the generation of genetically modified SAK compositions exhibiting enhanced anti-tumor activities.

Thus, provided in embodiments of the invention are cell compositions for ACT, characterized by improved therapeutic properties. In other embodiments, the invention relates to improved processes for producing cell compositions for ACT. According to some embodiments, the cell compositions of the invention may advantageously be produced directly from PBMC samples by ex vivo expansion protocols as disclosed herein, without a preceding step of apheresis or other purification steps, and without the need to isolate tumor-specific cells from tumor biopsies. In some embodiments, the cell compositions of the invention are produced by a process in which the cells of the sample are expanded by incubation or culturing in the presence of IL-2 and a CD3 activator (e.g. CD3-specific stimulating antibody) for at least 9 days and typically for 10-16 days, e.g. 10-12 days. According to advantageous embodiments as disclosed herein, the cells are expanded in the constant presence of effective amounts of the IL-2 and CD3 activator, such that effective amounts of said IL-2 and CD3 activator are periodically supplemented to the cells throughout the expansion period (typically every 2-4 days).

In one aspect, there is provided an ACT cell composition, comprising in vitro-expanded PBMC (e.g. at least 5×10⁶-10×10⁸ viable cells), of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo.

In one embodiment, 7-12% of the cells are CD3⁺CD4⁺ cells, 20-28% are CD3⁺CD56⁺ cells, and 10-14% are CD3⁺CD8⁺CD56⁺ cells. In another embodiment, the amount of CD3⁺CD8⁺ cells in the composition is 6-8 times the amount of the CD3⁺CD4⁺ cells in the composition. In an exemplary embodiment, the cell composition is characterized by a ratio of CD3⁺CD8⁺ cells to CD3⁺CD4⁺ cells of 7-8. In another embodiment, the composition comprises less than 3% CD3⁻CD56⁺ cells. In another embodiment, at least 40% of the CD3⁺CD8⁺ cells are characterized by surface expression of at least one marker selected from the group consisting of perforin, Fas-ligand (FasL), CXCR4 and CXCR2. In another embodiment, said CD3⁺CD8⁺ cells are further characterized as DNAM-1⁺, CTLA-4⁻ cells. In another embodiment, at least 90% of said CD3⁺CD8⁺ cells are characterized by surface expression of CXCR3 and at least 30% of said CD3⁺CD8⁺ cells are characterized by surface expression of CXCR6. In another embodiment, no more than 50% of the CD3⁺CD8⁺ cells are characterized by surface expression of a plurality of immune checkpoint molecules selected from the group consisting of CTLA-4, PD-1, and TIGIT.

In another embodiment, the cell composition exhibits non-MHC restricted cytotoxic activity mediated by granzyme B and/or perforin against tumor cells. In another embodiment, the cell composition exhibits non-MHC restricted cytotoxic activity mediated by granzyme B and/or perforin against hematopoietic tumor cells and solid tumor cells. In another embodiment, said cytotoxic activity is not substantially mediated by FAS-FasL interactions. In another embodiment, the cell composition is capable of inhibiting or preventing tumor development in a non-histocompatible subject in vivo without substantially eliciting GVHD.

In another embodiment the cell composition is capable of specifically eradicating hematopoietic tumor cells in vitro at a ratio of 0.25:1 within 24 hours. In another embodiment, the cell composition is capable of specifically eradicating hematopoietic tumor cells in a significant manner when incubated with the tumor cells in vitro at a ratio of 0.25:1 for 24 hours, and/or of specifically eradicating hematopoietic tumor cells such that 80-90% of the tumor cells are eradicated upon incubation in vitro for 24 hours at a ratio of composition cells to tumor cells of 1:1. In another embodiment, the tumor cells express at least one NKG2D ligand. In another embodiment, said tumor cells express a plurality of NKG2D ligands selected from the group consisting of: MICA, MICB, HLAE, ULBP1, ULBP256, and ULBP3. In another embodiment, the cell composition comprises 5×10⁶-10×10⁹ viable cells.

In another embodiment, the cell composition is prepared by a process comprising: expanding PBMC in the presence of effective amounts of IL-2 and a CD3 activator for at least nine days, wherein effective amounts of the IL-2 and CD3 activator are supplemented every 48-96 hours. In another embodiment, the incubation is performed in the absence of other cytokines or antibodies.

In another embodiment, the cells of the cell composition are genetically modified. In another embodiment, the cells are genetically modified to express at least one of a chimeric antigen receptor (CAR), cytokine, chemokine, a cytokine or chemokine receptor, or any combination thereof. In another embodiment, the cell composition is genetically modified to expresses a CAR directed to an antigen selected from the group consisting of: BCMA, CD47, PDL-1, mesothelin, EpCAM, CD34, CD44, PSCA, MUC16, CD276, CD123, CD19, CD20 and EFFRvIII. In another embodiment, the cell composition any one of a CD19-specific CAR and a CD123-specific CAR. In another embodiment, the cells are genetically modified to express a modified CXCR4 receptor comprising a mutation or truncation at the C-terminal tail domain. In another embodiment, the cell composition expresses any one of exogenous IL-2, exogenous IL-15, exogenous IL-21, or any combination thereof. In another embodiment, the cell composition is modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21.

In another aspect, there is provided a process for producing a cell composition for ACT, comprising:
a. provided a PBMC sample,
b. expanding the PBMC in the constant presence of effective amounts of IL-2 and a CD3 activator for at least nine days, and in the absence of other cytokines or antibodies, wherein effective amounts of the IL-2 and CD3 activator are supplemented every 48-96 hours,
wherein the number of viable cells in the culture is enhanced by at least 20-fold, and a cell composition comprising at least 5×10⁶-10×10⁸ viable cells is obtained.

In another embodiment, expansion is performed in the constant presence of said IL-2 and CD3 activator as sole exogenously-added cell stimulators. In another embodiment, step b. comprises expanding the cells in the presence of 500-1500 IU/ml IL-2 and 10-60 ng/ml anti-CD3 antibody, which are re-supplemented every 48-96 hours, for between 10-16 days. In another embodiment, the cells are not subjected to additional enrichment, stimulation or expansion steps. In another embodiment, the expansion is performed so as to enhance the number of viable cells by at least 30-fold. In another embodiment, expansion is performed so as to produce a cell composition comprising 5×10⁶ to 10×10⁹ viable mononuclear cells, of which 70-85% are CD3⁺CD8⁺ cells expressing NKG2D and granzyme B. In another embodiment, expansion is performed so as to produce a cell composition capable of specifically eradicating hematopoietic tumor cells in a significant manner when incubated with the tumor cells in vitro at a ratio of 0.25:1 for 24 hours. In another embodiment, the resulting cell composition is capable of specifically eradicating hematopoietic tumor cells in vitro at a ratio of 0.25:1 within 24 hours. In another embodiment, step (b.) further comprises genetically modifying the cells. In another embodiment, the process comprises genetically modifying said cells to express at least one of a CAR, a cytokine, a chemokine, a receptor of a cytokine or chemokine, or any combination thereof. In another embodiment, the process comprises modifying the cells to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. In another embodiment, the process further comprises cryopreserving the obtained cell composition.

In another embodiment, there is provided a cell composition prepared by the process. In another embodiment, there is provided a cell composition for ACT, prepared by a process comprising:
a. providing a PBMC sample,
b. expanding the PBMC in the constant presence of effective amounts of IL-2 and a CD3 activator for at least 9 days, and in the absence of other cytokines or antibodies, wherein effective amounts of the IL-2 and CD3 activator are supplemented every 2-4 days, so as to enhance the number of viable cells in the culture by at least 20-fold, to thereby obtain a cell composition comprising at least 5×10⁶-10×10⁸ viable cells, and
c. collecting the resulting cell composition.

In another aspect, there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21.

In another embodiment, 70-85% of the cells are CD3⁺CD8⁺ cells expressing NKG2D and granzyme B, at least 70% are CD56⁻ cells, and up to 5% are CD3⁻ cells. In another embodiment, the cell composition is prepared by a process comprising expanding PBMC in the constant presence of effective amounts of IL-2 and a CD3 activator for at least nine days, wherein effective amounts of the IL-2 and CD3 activator are supplemented 48-96 hours.

In another embodiment, there is provided a cell composition of the invention for use in treating a tumor in a subject in need thereof. In another embodiment, there is provided an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, for use in treating a tumor in a subject in need thereof. In another embodiment there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21, for use in treating a tumor in a subject in need thereof. In another embodiment, there is provided a cell composition prepared by a process as disclosed herein, for use in treating a tumor in a subject in need thereof.

In various embodiments, the tumor is a hematopoietic tumor or a solid tumor. In another embodiment, said tumor is selected from the group consisting of leukemia, multiple myeloma, a prostate tumor or mesothelioma. In another embodiment, said tumor is characterized by expression of at least one NKG2D ligand. In another embodiment, said tumor expresses a plurality of NKG2D ligands selected from the group consisting of: MICA, MICB, HLAE, ULBP1, ULBP256, and ULBP3. In another embodiment, said tumor expresses MICA, MICB, HLAE, ULBP1, ULBP256, and ULBP3. In another embodiment, said tumor is characterized by expression of at least one chemokine selected from the group consisting of CXCR6 ligands, CXCR4 ligands, CXCR2 ligands and CXCR3 ligands.

In another embodiment, said tumor is characterized by expression of at least one ligand of an inhibitory immune checkpoint molecule selected from the group consisting of CTLA-4, PD-1, and TIGIT. In another embodiment, said tumor is resistant to treatment by at least one checkpoint molecule inhibitor. In another embodiment, said tumor is resistant to treatment by at least one CTLA-4-specific blocking antibody. In another embodiment, said subject is not under a treatment regimen with checkpoint molecule inhibitors. In another embodiment, the use further comprises administering to said subject at least one checkpoint molecule inhibitor directed to Lag-3, Tim-3, or combinations thereof.

In another embodiment, said cell composition has undergone cryopreservation and thawing. In various embodiments, the cell composition is autologous, histocompatible allogeneic, or non-histocompatible allogeneic to said subject. In a particular embodiment, said cell composition is non-histocompatible allogeneic to said subject. In another embodiment, said tumor is characterized by down-regulation of MHC I expression and/or activity.

In another aspect, there is provided a method of treating a tumor in a subject in need thereof, comprising contacting the tumor cells with a therapeutically effective amount of a cell composition of the invention, thereby treating the tumor in the subject. In another embodiment there is provided a method of treating a tumor in a subject in need thereof, comprising contacting the tumor cells with a therapeutically effective amount of an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, thereby treating the tumor in the subject. In another embodiment. there is provided a method of treating a tumor in a subject in need thereof, comprising contacting the tumor cells with a therapeutically effective amount of a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21, for use in treating a tumor in a subject in need thereof, thereby treating the tumor in the subject. In another embodiment there is provided a method of treating a tumor in a subject in need thereof, comprising contacting the tumor cells with a therapeutically effective amount of a cell composition prepared by a process as disclosed herein, thereby treating the tumor in the subject.

In various embodiments, the cell composition is autologous, histocompatible allogeneic, or non-histocompatible allogeneic to said subject. In a particular embodiment, said cell composition is non-histocompatible allogeneic to said subject. In another embodiment, the contacting is performed in vivo. In another embodiment, the contacting is performed ex vivo. In another embodiment, said cell composition has undergone cryopreservation and thawing prior to contacting with said tumor cells. In another embodiment, the tumor is a hematopoietic tumor. In another embodiment, said tumor is a solid tumor. In another embodiment, said tumor is selected from the group consisting of leukemia, multiple myeloma, a prostate tumor or mesothelioma. In another embodiment, said tumor is characterized by expression of at least one NKG2D ligand. In another embodiment, said tumor expresses a plurality of NKG2D ligands selected from the group consisting of: MICA, MICB, HLAE, ULBP1, ULBP256, and ULBP3. In another embodiment, said tumor expresses MICA, MICB, HLAE, ULBP1, ULBP256, and ULBP3.

In another embodiment, said tumor is characterized by expression of at least one chemokine selected from the group consisting of CXCR6 ligands, CXCR4 ligands, CXCR2 ligands and CXCR3 ligands. In another embodiment, said tumor is characterized by expression of at least one ligand of an inhibitory immune checkpoint molecule selected from the group consisting of CTLA-4, PD-1, and TIGIT. In another embodiment, said tumor is resistant to treatment by at least one checkpoint molecule inhibitor. In another embodiment, said tumor is resistant to treatment by at least one CTLA-4-specific blocking antibody. In another embodiment, said subject is not under a treatment regimen with checkpoint molecule inhibitors. In another embodiment, the use further comprises administering to said subject at least one checkpoint molecule inhibitor directed to Lag-3, Tim-3, or combinations thereof. In another embodiment, said tumor is characterized by down-regulation of MHC I expression and/or activity.

Other objects, features and advantages of the present invention will become clear from the following description and drawings.

### BRIEF DESCRIPTION OF THE DRAWINGS

FIGS. 1A-1D. show a characterization of super-activated killer (SAK) cells at different time points during expansion (days 0, 4, 6, 8 and 11). FIG. 1A—number of total viable cells (×10⁶); FIGS. 1B-1D—flow cytometry analyses of the proportion (%) of CD3⁺CD8⁺ cells, CD3⁺CD4⁺ cells, and CD3⁺CD56⁺ cells, respectively.

FIGS. 2A-2B. Cytotoxic activity of SAK cells at different ratios of target:effector cells against hematopoietic tumor cells. FIG. 2A—effect against leukemia (THP-1) cells. FIG. 2B—effect against multiple myeloma (RPMI 8266) cells. The proportion (%) of live tumor cells after 24 hr incubation (CFSE⁺PI⁻) was evaluated by FACS.

FIGS. 3A-3C. Cytotoxic activity of SAK cells at different ratios of target:effector cells against hematopoietic and solid tumor cells. FIG. 3A—effect against leukemia MV4-11 cells FIG. 3B—effect against prostate cancer PC3 cells FIG. 3C—effect against mesothelioma cancer H28 cells.

FIGS. 4A-4B. Superior cytotoxic activity of SAK cells compared to other effector cells against leukemia MV4-11 cells, at different ratios of target:effector cells. FIG. 4A—effect compared to cytokine-induced killer cells (“CIK IFN”) FIG. 4B—effect compared to activated lymphocyte compositions (“T cells IL-2”).

FIG. 5. Expression of NKG2D receptor on cytotoxic CD8⁺ SAK cells.

FIGS. 6A-6C. Expression of effector molecules by SAK cells. FIG. 6A—FasL expression; FIG. 6B—Granzyme B expression; FIG. 6C—perforin expression.

FIG. 7. Perforin secretion by SAK cells following 24-hour incubation with leukemia MV4-11 cells.

FIGS. 8A-5C. show evaluation of the cytotoxic capacity in the presence of various inhibitors. SAK cells were incubated with MV4-11 cells at different ratios of target:effector cells in the presence of various inhibitors. FIG. 8A—granzyme B inhibitor (DCI); FIG. 8B—anti FasL antibody; FIG. 8C—perforin inhibitor.

FIGS. 9A-9B. show expression and function of chemokine receptors on CD8⁺ SAK cells at day 11 of expansion. FIG. 9A—expression of CXCR2, CXCR3 (top panel, left and right, respectively), CXCR4 and CXCR6 (bottom panel, left and right, respectively), as indicated by; black line indicates staining with isotype control antibodies (IC) conjugated to the respective fluorescent markers (FITC, APC or PE). FIG. 9B—transwell migration of SAK cells towards the chemokines IL-8, SDF-1 and IP-10.

FIGS. 10A-10C. show the inhibition of tumor cell engraftment in NSG mice following transfer of CD19⁺ SAK cells, compared to controls, measured as the percentage of CD19⁺ Raji cells. FIG. 10A—engraftment in spleen; FIG. 10B—engraftment in bone marrow (BM); FIG. 10C—engraftment in blood.

FIGS. 11A-11B. Illustrate the cytotoxic immune response induced by CD19 CAR SAK cells at different target:effector ratios. FIG. 11A—cytotoxic activity of SAK cells and CD19 CAR SAK cells against CD19⁺ tumor cells (Toledo). FIG. 11B—cytotoxic activity of SAK cells and CD19 CAR SAK cells against CD19—tumor cells (U937).

FIGS. 12A-12C. exemplify that SAK cells induced much less graft versus host disease (GVHD) in NSG mice compared with PBMC. Irradiated NSG mice received SAK cells or PBMC at dose of 5×10⁶. FIG. 12A—percentages of survival; FIG. 12B—body weight change; FIG. 12C—averaged GVHD scoring (“Ave Scoring”).

FIGS. 13A-13B. show the cytotoxic activity of SAK cells following a freeze-thaw cycle (denoted by “frozen SAK”), against THP-1 or MV411 tumor cells, in different SAK-tumor cell ratios, compared to fresh SAK cells which have not been frozen, denoted by “SAK”. FIG. 13A—activity against THP-1 cells. FIG. 13B—activity against MV411.

FIG. 14. depicts the levels of the following immune checkpoint molecules on the surface of CD8⁺ SAK cells: Tim-3 (top panel, left), DNAM-1 (top panel, right), PD-1 (middle panel, left), TIGIT (middle panel, right), Lag-3 (bottom panel, left) or CTLA-4 (bottom panel, right). APC or PE-labeled non-specific antibodies were used as isotype controls (IC).

FIG. 15. shows evaluation of the involvement of MHC I in SAK-mediated leukemic cell lysis. The cytotoxic effect against MV411 cells, in the presence (“anti-HLA”) or absence (“control”) of an anti-MHC I neutralizing antibody, is shown as a percentage of live tumor cells.

### DETAILED DESCRIPTION OF THE INVENTION

The invention relates to improved cell compositions and methods for adoptive cell therapy (ACT), useful in the treatment of cancer. More specifically, embodiments of the invention employ the use of cell compositions, herein designated super-activated killer cells (SAK), characterized by enhanced cytotoxicity, including against non-histocompatible tumor cells. The invention further relates to processes for the generation of SAK compositions, and to their use in cancer management. According to advantageous embodiments, the invention further relates to genetically modified SAK compositions, exhibiting improved survival and/or enhanced tumor engagement and retention.

The invention is based, in part, on the development of SAK compositions, capable of exerting anti-tumor cytotoxic activity against a variety of tumor cells in a highly potent manner, while not requiring MHC recognition. Surprisingly, SAK cells, produced from peripheral blood mononuclear cells (PBMC) by a unique culturing process as disclosed herein, were significantly more effective at eradicating tumor cells than other PBMC-derived effector cell compositions, including cytokine-induced killer cells (CIK) and interleukin-2 (IL-2)-expanded T cell compositions, produced according to hitherto known ACT protocols. Further, the generated SAK cells were found to be exceptionally effective in inhibiting tumor development in vivo, to exhibit improved safety, manifested as a markedly reduced risk of invoking graft-versus-host disease (GVHD), and to be compatible with cryopreservation. In addition, the cells were demonstrated to display an advantageous profile of cell-surface receptors, including immune checkpoint molecules and chemokine receptors. In particular, SAK cells were found to be characterized by high expression of activating receptors including DNAM-1, and minimized expression of inhibitory checkpoint molecules (e.g. CTLA-4). The invention is also based, in part, on the development of genetically modified SAK cell compositions, amenable for improved therapeutic modalities involving enhanced and/or prolonged tumor-specific activity.

Thus, provided in embodiments of the invention are cell compositions for ACT, characterized by improved therapeutic properties. In other embodiments, the invention relates to improved processes for producing cell compositions for ACT. According to some embodiments, the cell compositions of the invention may advantageously be produced directly from PBMC samples by ex vivo expansion protocols as disclosed herein, without a preceding step of apheresis or other purification steps, and without the need to isolate tumor-specific cells from tumor biopsies. In some embodiments, the cells of the sample (e.g. between 0.25×10⁶ to 5×10⁶ PBMC) are expanded by incubation or culturing in the presence of IL-2 and a CD3 activator (e.g. CD3-specific stimulating antibody) for at least 9 days and typically for 9-16 days, more typically 10-12 days. It is to be understood, that the anti-CD3 antibody may conveniently be used in a free suspension form, and need not be plate bound or presented by allogeneic feeder cells.

In particular, the cells are advantageously expanded in the constant presence of effective amounts of said IL-2 and CD3 activator, such that they are maintained at sufficient amounts throughout the entire expansion period (e.g. 500-1500 IU/ml IL-2 and 20-40 ng/ml anti-CD3 antibody for at least 9 days). Thus, according to the teachings of the invention, the constant presence of said stimulators is maintained by periodically supplementing the culture medium with said IL-2 and CD3 activator every 1-5 days and typically every 2-4 days. For example, the PBMC may be cultured at a concentration of about 1×10⁶ cells/ml in the presence of initial concentrations of about 1000 IU/ml recombinant human IL-2 (rhIL-2) and about 30 ng/ml anti human CD3 antibody (e.g. OKT-3), which are re-supplemented every 2-4 days during culture (e.g. at days 0, 4, 6 and 8).

According to additional advantageous embodiments, expansion is performed in the presence of said IL-2 and CD3 activator as sole cell stimulators. Thus, in some embodiments, expansion is performed in the absence of additional exogenously added cell stimulators, such as cytokines (e.g. IFN-7) and/or of feeder cells. In another embodiment, the process is performed so as to enhance the number of viable cells in the culture by at least 20-fold and typically by at least 30-fold on average (e.g. 30-55-fold). In another embodiment, the process provides 25-55-fold enhancement in viable cell counts within 11 days of culture. In another embodiment the process is performed so as to produce at least 1.5×10⁸ and typically 2×10⁸ to 4×10⁸ viable cells, most typically 5×10⁶ to 10×10⁸ viable cells and up to about 10×10⁹ viable cells.

In one aspect, there is provided an ACT cell composition, comprising in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo.

In another aspect, there is provided a process for producing a cell composition for ACT, comprising:
a. provided a PBMC sample,
b. expanding the PBMC in the constant presence of effective amounts of IL-2 and a CD3 activator for at least nine days, and in the absence of other cytokines or antibodies, wherein effective amounts of the IL-2 and CD3 activator are supplemented every 48-96 hours,
   wherein the number of viable cells in the culture is enhanced by at least 20-fold, and a cell composition comprising at least 5×10⁶-10×10⁸ viable cells is obtained.

In another embodiment, there is provided a cell composition prepared by the process. In another embodiment there is provided a cell composition for ACT, prepared by a process comprising:
a. providing a PBMC sample,
b. expanding the PBMC in the constant presence of effective amounts of IL-2 and a CD3 activator for at least 9 days, and in the absence of other cytokines or antibodies, wherein effective amounts of the IL-2 and CD3 activator are supplemented every 2-4 days, so as to enhance the number of viable cells in the culture by at least 20-fold, to thereby obtain a cell composition comprising at least 5×10⁶-10×10⁸ viable cells, and
c. collecting the resulting cell composition.

In another aspect, there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21.

In another embodiment, there is provided a cell composition of the invention for use in treating a tumor in a subject in need thereof. In another embodiment, there is provided an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, for use in treating a tumor in a subject in need thereof. In another embodiment there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21, for use in treating a tumor in a subject in need thereof. In another embodiment, there is provided a cell composition prepared by a process as disclosed herein, for use in treating a tumor in a subject in need thereof.

In another aspect, there is provided a method of treating a tumor in a subject in need thereof, comprising contacting the tumor cells with a therapeutically effective amount of a cell composition of the invention, thereby treating the tumor in the subject. In another embodiment there is provided a method of treating a tumor in a subject in need thereof, comprising contacting the tumor cells with a therapeutically effective amount of an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, thereby treating the tumor in the subject. In another embodiment. there is provided a method of treating a tumor in a subject in need thereof, comprising contacting the tumor cells with a therapeutically effective amount of a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21, for use in treating a tumor in a subject in need thereof, thereby treating the tumor in the subject. In another embodiment there is provided a method of treating a tumor in a subject in need thereof, comprising contacting the tumor cells with a therapeutically effective amount of a cell composition prepared by a process as disclosed herein, thereby treating the tumor in the subject.

These and other aspects and embodiments are described in further detail and exemplified below.

### Preparation Process

In some embodiments, the invention relates to a process for producing a cell composition of the invention. In some embodiments, provided is a process for producing a cell composition for ACT, comprising:
a. providing a PBMC sample,
b. expanding the PBMC in the constant presence of effective amounts of IL-2 and a CD3 activator for at least 9 days, and in the absence of other cytokines or antibodies, wherein effective amounts of the IL-2 and CD3 activator are supplemented every 2-4 days, so as to enhance the number of viable cells in the culture by at least 20-fold, to thereby obtain a cell composition comprising at least 5×10⁶ to 10×10⁸ viable cells, and
c. collecting the resulting cell composition.

In another embodiment, the process comprises:
a. provided a PBMC sample,
b. expanding the PBMC in the constant presence of effective amounts of IL-2 and a CD3 activator for at least nine days, and in the absence of other cytokines or antibodies, wherein effective amounts of the IL-2 and CD3 activator are supplemented every 48-96 hours,
wherein the number of viable cells in the culture is enhanced by at least 20-fold, and a cell composition comprising at least 5×10⁶-10×10⁸ viable cells is obtained.

The term “PBMC sample” or “peripheral blood mononuclear cells sample” or “un-fractionated PBMC”, as used herein, refers to whole PBMC, i.e., to a population of viable white blood cells having a round nucleus, which has not been substantially enriched in a given sub-population. Typically, the PBMC sample according to the invention has been obtained from peripheral blood and has not been subjected to a selection step to contain only adherent PBMC (which consist essentially of >90% monocytes) or non-adherent PBMC (which contain T cells, B cells, natural killer (NK) cells, NK T cells and DC precursors).

Typically, PBMC can be obtained (e.g. extracted or partially purified from peripheral blood) by centrifugation (e.g., from a buffy coat), by density gradient centrifugation (e.g., through a Ficoll-Hypaque) or by other methods known in the art. For example, PBMC may be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

The term “expanding” as used herein refers to increasing the number of cells of a cell population due to cell replication. In particular, PBMC expansion as disclosed herein refers to an in vitro or ex vivo culturing process comprising polyclonal activation and multiplication of cell populations within the PBMC, so as to produce an ACT cell composition in accordance with the invention. Typically, e.g. for producing a clinical-grade ACT composition for treatment of a human subject, expansion is performed in a specific cGMP grade environment and cGMP grade medium. In some embodiments, expansion is performed for 9-16 days or 11-16 days, e.g. 9, 10, 11, 12, 13, 14, 15, or 16 days.

More specifically, interleukin-2 (IL-2) and a CD3 activator are supplemented to the culture medium during the expansion process. In some embodiments, the cell composition is prepared by a process as described above, wherein effective amounts of IL-2 and CD3 activator are supplemented every 48-96 hours, e.g. every 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours.

The cytokine IL-2 acts as a growth factor for lymphocytes in culture, and is required for example for preventing cell death of activated T cells. The activity of IL-2 is mediated by binding to and signaling through the IL-2 receptor (IL-2R), a complex consisting of three chains, termed alpha (CD25), beta (CD122) and gamma (CD132). Dimeric IL-2R is expressed by memory CD8⁺ T cells and NK cells, whereas regulatory T cells and activated T cells express high levels of trimeric IL-2R. As used herein, “interleukin 2” or “IL-2” refers to mammalian IL-2, preferably human IL-2 (e.g. recombinantly produced human IL-2). In some embodiments, polypeptides recognized in the art as biological equivalents of human IL-2, capable of selectively binding to IL-2 receptors and exerting comparable activity in supporting the expansion and proliferation of human lymphocytes (e.g. IL-2 fusion proteins, conjugates and variants) may be used. IL-2 may be produced recombinantly (e.g. by methods as disclosed herein) and is commercially available from a variety of sources. For example, recombinant human IL-2 (rhIL-2) may be purchased from R&D Systems. Proleukin® (aldesleukin) is a highly purified rhIL-2 protein, approved for clinical use in cancer immunotherapy.

The CD3 complex is associated with T cell receptor (TCR) chains, forming the TCR-CD3 complex on the surface of T cells. Thus, CD3 is considered a T cell marker, although certain other cell populations such as NK-T cells, are also characterized by CD3 surface expression (CD3⁺ cells), whereas other cell types, such as NK cells, B cells and myeloid cells are not generally characterized by lack of CD3 surface expression (CD3⁻ cells). In mammals, the TCR-CD3 complex is comprised of a CD37 chain, a CD36 chain, and two CD3ε chains, complexed with the TCR chains and the CD3-zeta (ζ-chain). As used herein, a “CD3 activator” is an agent capable of inducing or mediating polyclonal stimulation and proliferation by binding to a CD3 molecule on the surface of a lymphocyte, typically, a human CD3 molecule. Examples of CD3 activators include stimulatory CD3-specific antibodies, antigen-binding portions thereof, and conjugates thereof. Typically, the CD3 activator is a stimulatory anti-CD3 monoclonal antibody (mAb), typically directed to a CD3 signaling domain such as on CD3ε. In a particular embodiment, an advantageous CD3 activator to be used in embodiments of the invention is OKT3, a murine monoclonal antibody directed against the human CD3ε chain. Other exemplary antibodies that crosslink the T cell receptor/CD3 complex include the HIT3a and UCHT1 anti-CD3 mAbs.

Additional examples of CD3 activators are CD3 ligands and CD3-binding mitogens. Such antibodies and agents are commercially available from a variety of sources. For example, OKT3 and other anti-CD3 mAbs are available from Biolegend and eBioscience. According to embodiments of the invention, the PBMC are expanded in the absence of other cytokines or antibodies. In other words, cytokines and antibodies other than IL-2 and the CD3 activator are not supplemented (added exogenously) to the culture. Therefore, expansion processes in accordance with the invention are distinguishable from other expansion protocols, used for producing other types of cell compositions (for example CIK cells are produced by expansion of e.g. PBMC in the presence of inter alia IFN-γ). It is to be understood, however, that during the expansion process, various factors, including cytokines, may be produced by the cultured cells and secreted to the culture media.

Further, expansion processes in accordance with the invention are performed in the constant presence of said IL-2 and CD3 activator, such that they are supplemented at either a continuous or intermittent manner such that they are maintained at effective amounts throughout the expansion process. Therefore, expansion processes in accordance with the invention are distinguishable from other expansion protocols, used for producing other types of cell compositions (for example, the production of CIK cells involves addition of IL-2 and OKT3 on the second day of expansion, wherein OKT3 is not subsequently supplemented to the culture).

In other embodiments, expansion is performed in the constant presence of said IL-2 and CD3 activator as sole exogenously-added cell stimulators. Thus, other cell stimulators used to induce lymphocyte activation and proliferation during in vitro or ex-vivo expansion processes, such as antibodies directed to co-stimulatory molecules (e.g. CD28), antigens (e.g. MHC-antigen complexes) and the like, which are of an exogenous source to the cultured cells, are not artificially introduced (supplemented). It is to be understood, that certain cell stimulators may be produced endogenously by the cultured cells during the expansion process. It is further understood, that various factors included in standard tissue culture media (for example, in complete medium such as RPMI) are not considered to be exogenously-added cell stimulators according to these embodiments. For example, expansion may conveniently be performed in the presence of tissue culture serum, e.g. fetal calf serum (FCS) at a final concentration of 5-15%, typically 7-12% or 8-13% and more typically about 10%.

As disclosed herein, expansion is advantageously performed so as to enhance the number of viable cells in the culture by at least 20-fold (or in some embodiments 30-60-fold), to thereby obtain a cell composition comprising 5×10⁶ to 10×10⁸ or in other embodiments up to 10×10⁹ viable cells. The term “viable cells”, as used herein, refers to cells not undergoing necrosis or cells which are not in an early or late apoptotic state. Assays for determining cell viability are known in the art, such as using propidium iodide (PI) staining which may be detected by flow cytometry. Accordingly, in one embodiment, viable cells are cells which do not show propidium iodide intake and do not express phosphatidylserine. Necrosis can be further identified, by using light, fluorescence or electron microscopy techniques, or via uptake of the dye trypan blue.

In addition, the production processes of the invention comprise a step of collecting the cell composition resulting from the expansion step. For example, the cells may be harvested and subjected to centrifugation or other washing steps, to remove residual antibodies and cytokines. In some embodiments, cells may be re-suspended in a suitable diluent or vehicle (e.g. PBS) prior to contacting with tumor cells or administration to a subject.

In some embodiments, the cells have not been subjected to additional steps of enrichment (e.g. apheresis, lymphocytoapheresis, or immunocapture of leukocytes prior to the expansion step), stimulation (e.g. antigen-specific) and/or expansion (e.g. a rapid expansion protocol in the presence of feeder cells or propagation in the presence of other/additional cytokines).

In another aspect, there is provided a process for producing a cell composition of the invention, the process comprising:
a) providing a PBMC sample,
b) expanding the PBMC in the constant presence of effective amounts of IL-2 and a CD3 activator for at least 9 days, wherein effective amounts of the IL-2 and CD3 activator are supplemented every 2-4 days, and
c) collecting the resulting cell composition.

In some embodiments, expansion is performed so as to obtain a cell composition of the invention as disclosed herein. In another embodiment expansion is performed in the constant presence of said IL-2 and CD3 activator as sole exogenously-added cell stimulators (in the absence of other cytokines and antibodies).

In another embodiment the method further comprises genetically modifying the cells to obtain a cell composition as disclosed herein. In some embodiments, the resulting cell composition is advantageously amenable for cryopreservation. Thus, as opposed to certain other cell compositions for ACT, the cell compositions of the invention may be frozen following expansion and thawed prior to administration to the subject, without substantial loss of potency. Thus, in another embodiment, the process further comprising freezing the resulting cell composition. For example, without limitation, a cryopreservation protocol may be performed using liquid nitrogen, wherein the cells are suspended in suitable media (e.g. containing 90% FCS)+10% Dimethyl sulfoxide (DMSO)). Optionally, commercially available systems or excipients (e.g. CryoSure® USP grade cryo-protective agents).

In another embodiment, step b. comprises expanding the cells in the presence of 500-1500 IU/ml IL-2 and 10-60 ng/ml anti-CD3 antibody, which are re-supplemented every 2-4 days, for 10-16 days. In another embodiment the cells have not been subjected to additional enrichment, stimulation or expansion steps (e.g. before or after step b.). In another embodiment wherein the expansion is performed so as to enhance the number of viable cells by at least 30-fold. In another embodiment expansion is performed so as to produce a cell composition comprising at least 5×10⁶ to 10×10⁸ (e.g. 5×10⁶ to 10×10⁹) viable mononuclear cells, of which 70-80% (or in other embodiments 70-85%) are CD3⁺CD8⁺ cells expressing NKG2D and granzyme B. In another embodiment expansion is performed so as to produce a cell composition capable of specifically eradicating hematopoietic tumor cells in a significant manner when incubated with the tumor cells in vitro at a ratio of 0.25:1 for 24 hours. In another embodiment step b. further comprises genetically modifying the cells, e.g. to produce a genetically modified cell composition as disclosed herein. Advantageously, the cells are modified (e.g. transduced or transfected) prior to subjecting the cells to supplementation of the CD3 stimulator and IL-2.

### Cell Compositions

In another embodiment there is provided a cell composition prepared by a process as disclosed herein. In another embodiment, there is provided a cell composition comprising SAK cells as disclosed herein. In another embodiment, the invention relates to adoptive cell transfer (ACT) compositions as disclosed herein. In another embodiment, there is provided an ACT cell composition, comprising 5×10⁶ to 10×10⁸ viable in vitro-expanded PBMC, of which 70-80% are CD3⁺CD8⁺ cells expressing NKG2D and granzyme B, at least 70% are CD56⁻ cells, and up to 5% are CD3⁻ cells, said composition exhibiting significant cytotoxic activity against non-histocompatible tumor cells in vitro and in vivo. In another embodiment, there is provided an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo.

As used herein, and unless otherwise specified, the term “adoptive transfer” refers to a form of passive immunotherapy where previously sensitized immunologic agents (e.g., cells or serum) are transferred to the recipients. The phrases “adoptive transfer immunotherapy”, “adoptive cell therapy” and “adoptive cell immunotherapy” are used interchangeably herein to denote a therapeutic or prophylactic regimen or modality, in which effector immunocompetent cells, such as the cell compositions of the invention, are administered (adoptively transferred) to a subject in need thereof, to alleviate or ameliorate the development or symptoms of cancer. Thus, an ACT composition in accordance of the invention contains effective amounts (e.g. at least 5×10⁶ cells and up to about 10×10⁹ cells), which are produced under sterile and suitable (e.g. cGMP grade) conditions, to be administered to a human subject afflicted with a neoplastic disorder as part of their anti-tumor regimen.

As used herein, the term “in vitro-expanded PBMC” refers to mononuclear cells that are of peripheral blood origin, which had undergone a step of expansion in vitro or ex vivo, resulting in a cell composition as disclosed herein. These cells and compositions are thus distinguishable from leukocyte compositions obtained or derived from other sources, such as tumor-derived lymphocytes (TIL, obtained from tumor biopsies rather than from peripheral blood), leukocyte cell lines, and leukocytes differentiated in vitro from pluripotent stem cells.

T lymphocytes (T cells) are one of a variety of distinct cell types involved in an immune response, and are characterized by surface expression of CD3 (CD3⁺ cells). The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell. Proper activation of T lymphocytes by antigen-presenting cells requires stimulation not only of the TCR, but the combined and coordinated engagement of its co-receptors.

T helper cells (TH cells) assist other white blood cells in immunologic processes, including maturation of B cells into plasma cells and memory B cells, and activation of cytotoxic T cells and macrophages. These cells are also known as CD4⁺ T cells (or CD3⁺CD4⁺ cells) because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response.

Cytotoxic T cells (Tc cells, or CTLs) destroy virus-infected cells and tumor cells, and are also implicated in transplant rejection. These cells are also known as CD8⁺ T cells (or CD3⁺CD8⁺ cells) since they express the CD8 glycoprotein at their surfaces. These cells recognize their targets by binding to antigen associated with MHC class I molecules, which are present on the surface of all nucleated cells.

Natural Killer cells (NK cells) are a subset of peripheral blood lymphocytes defined by the expression of CD56, CD16 and/or CD57 and the absence of the T cell receptor-CD3 complex. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release cytokines that stimulate or inhibit the immune response.

Natural killer T cells (NKT cells or NK-T cells) are CD1d-restricted T cells, which express a TCR. Unlike conventional T cells that detect peptide antigens presented by conventional MHC molecules, NK-T cells recognize lipid antigens presented by CD1d, a non-classical MHC molecule. Invariant or type I NK-T cells express a very limited TCR repertoire, whereas nonclassical or noninvariant type II NKT cells display a more heterogeneous TCRαβ usage. Adaptive or invariant (type I) NKT cells can be identified with the expression of at least one or more of the following markers, TCR Va24-Ja18, Vb11, CD1d, CD3, CD4, CD8, aGalCer, CD161 and CD56.

The neural cell adhesion molecule (NCAM), also known as CD56, is a member of the immunoglobulin superfamily engaged in both so-called homophilic and heterophilic interactions. Three main isoforms exist of CD56 (NCAM-120, NCAM-140, and NCAM-180), all generated by alternative splicing from one single gene, differing in their intracellular domain length. Expression of CD56 is most stringently associated with NK cells, which constitute the majority of CD56⁺ cells isolated from peripheral blood. However, CD56 has also been detected e.g. on other lymphoid cells, including but not limited to gamma delta (γδ) T cells and dendritic cells (DCs).

As used herein, a cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is specifically bound the cell. It is to be understood that while a cell may express messenger RNA for a cell-surface marker, in order to be considered positive for the compositions and methods described herein, the cell must express the marker of interest on its surface. Similarly, a cell is considered “negative” for a cell-surface marker if it does not express the marker on its surface in amounts sufficient to be detected using methods known to those of skill in the art, such as contacting a cell with an antibody that binds specifically to that marker and subsequently performing flow cytometric analysis of such a contacted cell to determine whether the antibody is bound the cell.

In various embodiments, the cell compositions of the invention are characterized by high relative CD8⁺ cell levels and low relative CD4⁺ cell levels. In some embodiments the compositions are characterized by an enhanced ratio of CD3⁺CD8⁺ cells to CD3⁺CD4⁺ cells (at least 6:1, e.g. 6.5-8.5:1, typically 7-8:1). In other words, the amount of CD3⁺CD8⁺ cells in the composition is 6-8 times and typically 7-8 times the amount of the CD3⁺CD4⁺ cells in the composition. In other embodiments the cell compositions are characterized by high NK-T cell levels. In other embodiments the cells are characterized by enhanced NK-T cells to T cells ratio. In some embodiments, the cell compositions are characterized by an incidence of CD3⁺CD8⁺CD56⁺ cells to CD3⁺CD8⁺ cells of 15-30%, typically 19-25 or 20-22%. In yet other embodiments, the cell compositions are characterized by enhanced ratios of CD8⁺ cells to CD56⁺ cells, e.g. 2-4. Such enhanced levels and ratios may relate in various embodiments to enhancement with respect to the original PBMC sample, or compared to other ACT compositions such as CIK cell preparations, as exemplified herein. Each possibility represents a separate embodiment of the invention.

For example, in some embodiments, the cell compositions of the invention are characterized by the presence of at least 60% and typically 75% on average (e.g. 70-80%, 70-85%, 73-76% or 73-78%) cytotoxic CD3⁺CD8⁺ cells. In another embodiment, the cell compositions are characterized by the presence of 70-85% are CD3⁺CD8⁺ cells. In other embodiments, the cell compositions are characterized by the presence of up to 15% (e.g. 7-12%, 11-13%, 9-12% or 8-11%) CD3⁺CD4⁺ cells, typically up to 10% on average CD3⁺CD4⁺ cells. In other embodiments, at least 15% of the CD3⁺CD8⁺ cells on average are CD8⁺CD56⁺ cells (e.g. 15-30% or 17-20%). In another embodiment the cell compositions are characterized by at least 9% and typically 10-14% CD3⁺CD8⁺CD56⁺ cells. In another embodiment the cell compositions are characterized by the presence of 7-17% CD3⁺CD8⁺CD56⁺ cells. It yet other embodiments, the cell compositions comprise about 8-35%, typically 20-28% or 22-26% CD3⁺CD56⁺ cells. In another embodiment, the cell compositions contain 15-30% CD3⁺CD56⁺ cells.

In some embodiments, the cell composition is characterized by high expression of activation markers and chemokine receptors. In some embodiments, the cells express at least one and typically a plurality of receptors selected from the group consisting of NKG2D, granzyme B, perforin, FASL, CXCR4 and CXCR2, wherein each possibility represents a separate embodiment of the invention. For example, as demonstrated herein, substantially all the CD8⁺ cells of the expanded cell composition were found to express activation markers NKG2D and granzyme B (see Examples 4 and 5 below). Thus, the invention in embodiments thereof provides improved cell compositions, in which the CD8⁺ cells are typically also characterized by NKG2D and granzyme B expression.

Natural killer Group 2 member D (NKG2D) is a transmembrane protein belonging to the NKG2 family of C-type lectin-like receptors. In humans, NKG2D is expressed by NK cells, 76 T cells and CD8⁺αβ T cells, acting as an activating receptor that promotes effector cell functions against target cells expressing NKG2D ligands. These ligands are induced-self proteins belonging to the MHC class I chain-relate (MIC) or UL-16-binding proteins (ULBP) families (e.g. MICA, MICB, HLAE, ULBP1, ULBP256, and ULBP3), which are completely absent or present only at low levels on surface of normal cells, and become overexpressed on infected, malignant, transformed, senescent and stressed cells. Their expression is regulated at different stages (by various stress pathway, most prominently the DNA damage response. NKG2D is associated with DNAX-activating protein 10 (DAP10), which promotes and stabilizes its surface membrane expression. NKG2D lacks a signaling motif in its cytoplasmic domain; signal transduction occurs upon ligation via the phosphorylation of DAP10, which recruits downstream signaling effector molecules and, ultimately, cytotoxicity.

According to additional embodiments, the CD8⁺ cells may further express perforin and/or FASL. In other embodiments, the CD8⁺ cells may further express CXCR4 and/or CXCR2. In other embodiments, the CD8⁺ cells may further express CXCR3 and/or CXCR6. Each possibility represents a separate embodiment of the invention. In another embodiment, at least 40% of the CD3⁺CD8⁺ cells are characterized by surface expression of at least one marker selected from the group consisting of perforin, FasL, CXCR4 and CXCR2. In another embodiment, at least 90% of said CD3⁺CD8⁺ cells are characterized by surface expression of CXCR3 and at least 30% of said CD3⁺CD8⁺ cells are characterized by surface expression of CXCR6.

CXC chemokine receptors are integral membrane proteins that specifically bind and respond to cytokines of the CXC chemokine family. Structurally, these receptors are characterized as G protein-linked receptors that are known as seven transmembrane (7-TM) proteins, since they span the cell membrane seven times.

CXCR4 (also known as fusin) is the receptor for a chemokine known as CXCL12 (or SDF-1) as its canonical ligand, a molecule endowed with potent chemotactic activity for lymphocytes. CXCR4 has a wide cellular distribution, with expression on various hematopoietic cell types (e.g. neutrophils, monocytes, T and B cells, dendritic cells, Langerhans cells and macrophages). CXCR4 activation can drive both cell migration and cell proliferation, and CXCL12 is also known to be important in hematopoietic stem cell homing to the bone marrow and in hematopoietic stem cell quiescence. Several additional ligands of CXCR4 have been disclosed, including high mobility group box 1 protein (HMGB1), which is a damage-associated molecular pattern molecule, macrophage migration inhibitory factor (MIF), a cytokine involved in the regulation of innate immunity, extracellular ubiquitin (eUb) and Beta-defensin-3 (HBD3).

CXCR2, also known as Interleukin 8 receptor, beta (IL8RB), is expressed on the surface of neutrophils and certain other cell types. CXCR2 binds with high affinity to IL-8 (also known as CXCL8), a chemokine produced by macrophages and other cell types such as epithelial cells, airway smooth muscle cells and endothelial cells, which is the primary cytokine involved in the recruitment of neutrophils to the site of damage or infection. Other ligands of CXCR2 include e.g. CXCL2, CXCL3, and CXCL5.

CXCR3, and its ligands, CXCL9, CXCL10, and CXCL11 are key immune chemoattractants during interferon-induced inflammatory responses. CXCR3 (GPR9/CD183) is an interferon-inducible chemokine receptor expressed on various cell types, but preferentially monocytes, Th1 T cells, CD8⁺ T cells, NKT cells, NK cells, dendritic cells, and some cancer cells. There are three isoforms of CXCR3 in humans: CXCR3-A, CXCR3-B and chemokine receptor 3-alternative (CXCR3-alt). CXCR3-A binds to the CXC chemokines CXCL9 (MIG), CXCL10 (IP-10), and CXCL11 (I-TAC) whereas CXCR3-B can also bind to CXCL4 in addition to CXCL9, CXCL10, and CXCL11.

CXCR6 (also known as STRL33 and TYMSTR) is a chemokine receptor which is expressed on e.g. naïve CD8⁺ cells, CD3⁻CD16^{-/low}CD56⁺ and CD3⁻CD16^{low}CD56⁻ NK cells, NKT cells, activated CD4⁺ and CD8⁺ T cells, and in 30-40% of γδ T cells. It binds exclusively to CXCL16 and plays a role in a range of immune processes including T cell recruitment in graft-versus-host disease, maintenance of liver-resident CD8 T cells following infection, mediating pulmonary disease severity and more. Its ligand is a chemotactic cytokine belonging to the α-chemokine subfamily which plays a significant role in tumor cell proliferation, migration, invasion, and metastasis. In particular, CXCR6 activation was suggested to be important for the survival and expansion of cytotoxic T cells at the tumor microenvironment, thereby reducing T cell exhaustion.

In addition, cell compositions in accordance with the invention are further characterized by surface expression of DNAM-1. In another embodiment, at least 90%, 93%, 95%, 97% and typically substantially all (up to 100%) of the CD3⁺CD8⁺ cells are characterized by surface expression of DNAM-1.

DNAX accessory molecule-1 (DNAM-1, CD226) is a transmembrane nectin-like glycoprotein of the immunoglobulin superfamily and acts as an activating receptor on NK cells and T cells. Its ligands, CD112 (also known as polio virus receptors (PVR)) and CD155 (also known as Nectin-2), are likewise members of the nectin family, and the DNAM-1-CD112/CD155 axis is mainly known for its important role in NK cell-mediated killing of tumor cells. it has been proposed that DNAM-1/PVR axis is involved in the NK cell-mediated lysis of allogeneic activated T cells, while in an autologous setting, NKG2D emerges as the dominant receptor.

In other embodiments, the cell compositions of the invention are characterized by reduced expression of inhibitory immune checkpoint molecules.

Immune checkpoint molecules represent an immune escape mechanism preventing the immune system from attacking its own body, by inducing self-tolerance. Typically, immune checkpoint receptors are present on T cells, and interact with immune checkpoint ligands expressed on antigen-presenting cells. However, some cancers can protect themselves from attack by stimulating immune checkpoint targets. T cells recognize an antigen presented on the MHC molecule and are activated to generate an immune reaction, whereas an interaction between immune checkpoint receptor and ligand that occurs in parallel with the above controls the activation of T cells.

Immune checkpoint receptors include co-stimulatory receptors and inhibitory receptors, and the T cell activation and the immune reaction are controlled by a balance between both receptors. As used herein, the term “inhibitory immune checkpoint molecules” include in particular inhibitory immune checkpoint receptors, expressed on immune effector cells such as T cells and capable of mediating a down-modulating or inhibiting an anti-tumor immune response upon engagement by their respective ligands. Examples include, without limitation, CTLA-4, PD-1, VISTA, B7-H3, B7-H4, HVEM, KIR family receptors, TIM-1, TIM-3, LAG-3, and TIGIT.

According to specific embodiments, CD3⁺CD8⁺ cells of the ACT compositions of the invention are characterized by reduced surface expression of one or more immune checkpoint molecules selected from the group consisting of CTLA-4, PD-1, TIGIT, Lag-3 and Tim-3. Such reduced surface levels may relate in various embodiments to reduction with respect to the original PBMC sample, or compared to other ACT compositions such as CIK cell preparations, as exemplified herein. Each possibility represents a separate embodiment of the invention.

In another embodiment, said CD3⁺CD8⁺ cells are characterized by lack of substantial surface expression of CTLA-4. In another embodiment, at least 95%, 97% and up to about 100% of said CD3⁺CD8⁺ cells are CTLA-4⁻ cells. Cytotoxic T Lymphocyte antigen 4 (CTLA-4 CD152) is a co-receptor that is constitutively expressed in regulatory T cells and is upregulated in conventional CD4⁺ and CD8⁺ T cells after activation—a phenomenon which is particularly notable in cancers. CTLA-4 binds CD80 and CD86 on APC with greater affinity and avidity than the activating receptor CD28, thus enabling it to outcompete CD28 for its ligands and promote T cell inhibition.

In another embodiment, no more than 25%, 20%, 15% or 10% of the CD3⁺CD8⁺ cells are characterized by surface expression of PD-1. In another embodiment, about 15% of the CD3⁺CD8⁺ cells are characterized by surface expression of PD-1. Programmed cell death protein 1 (PD1) is expressed by all T cells during activation as well as regulatory T cells, B cells, natural killer cells and some myeloid cell populations. PD1 often shows high and sustained expression levels during persistent antigen encounter, which can occur in the setting of chronic infections and cancer. In these settings, PD1 can limit protective immunity. PD1 has two ligands, PD-L1 and PD-L2, wherein expression of PD-L1 at the tumor microenvironment is commonly associated with immune evasion of the tumor.

In another embodiment, no more than 30%, 25%, 22%, 20% or 18% of the CD3⁺CD8⁺ cells are characterized by surface expression of TIGIT. In another embodiment, about 20-22% of the CD3⁺CD8⁺ cells are characterized by surface expression of TIGIT. T cell immunoglobulin and ITIM domain (TIGIT) is an inhibitory receptor expressed on NK cells and T cells, including CD4⁺ T cells, CD8⁺ T cells and T^{regs}. TIGIT expression is usually low in naive cells, but both T cells and NK cells have been shown to up-regulate TIGIT upon activation. TIGIT has three ligands, CD155 (polio virus receptors—PVR, NECL-5), CD112 and CD113, which all belong to a family of nectin and NECL molecules

In another embodiment, no more than 55%, 53%, 51%, 50% or 48% of the CD3⁺CD8⁺ cells are characterized by surface expression of Lag-3. In another embodiment, about 50-51% of the CD3⁺CD8⁺ cells are characterized by surface expression of Lag-3. Lymphocyte-activation gene 3 (LAG3, also known as CD223) is a member of the immunoglobulin superfamily expressed on activated T cells, TIL, T_{regs}, NK cells, B cells and plasmacytoid DC. LAG3 interacts with MHC-II to prohibit the binding of the same MHC molecule to TCR and CD4, thus directly hindering TCR signaling in immune response. Additional LAG3 ligands include FGL-1, α-synuclein fibrils (α-syn) and the lectins galectin-3 (Gal-3) and lymph node sinusoidal endothelial cell C-type lectin (LSECtin). LAG3 negatively regulates cellular proliferation, activation, and homeostasis of T cells, in a similar fashion to other immune checkpoint molecules, and has been reported to play a role in Treg suppressive function. For example, on T cells, LAG3 reduces cytokine and granzyme production and proliferation while encouraging differentiation into T regulatory cells.

In another embodiment, no more than 55%, 54%, 52%, 50% or 48% of the CD3⁺CD8⁺ cells are characterized by surface expression of Tim-3. In another embodiment, about 50-54% of the CD3⁺CD8⁺ cells are characterized by surface expression of Tim-3. T cell immunoglobulin and mucin domain-containing protein 3 (TIM3), a member of the TIM family, is expressed by interferon-γ (IFNγ)-producing CD4⁺ and CD8⁺ T cells, as well as many other cell types, including regulatory T cells, myeloid cells, NK cells and mast cells. TIM3 has been reported to have multiple different ligands (e.g. galectin 9, phosphatidylserine, carcinoembyronic antigen-related cell adhesion molecule 1 (CEACAM1) and high mobility group protein B1 (HMGB1)). TIM3 is part of a module that contains multiple co-inhibitory receptors (checkpoint receptors), which are co-expressed and co-regulated on dysfunctional or ‘exhausted’ T cells in chronic viral infections and cancer.

In another embodiment, the cell compositions exert a cytotoxic activity against tumor cells. In some embodiments, the tumor cells are of hematopoietic origin. The phrase “tumor cells of a hematopoietic origin” refers to malignant cells derived from blood cells or precursors thereof. In another embodiment the tumor cells are of lymphoid origin. In another embodiment the tumor cells are of myeloid origin. In a particular embodiment the tumor cells are leukemia cells, e.g. acute myeloid leukemia (AML) cells. In another particular embodiment, the tumor cells are multiple myeloma cells.

According to exemplary embodiments, the cell compositions are capable of specifically eradicating (killing) hematopoietic tumor cells when incubated with said cells in vitro at a ratio of at least 0.25:1 (respectively) for 24 hours. According to additional exemplary embodiments, the cell compositions are capable of specifically eradicating at least 50% and typically 60-99%, more typically about 80-90% of said hematopoietic tumor cells when incubated with said cells in vitro at a 1:1 ratio for 24 hours. In other embodiments, the tumor cells express at least one and typically a plurality of NKG2D ligands, including, but not limited to MICA, MICB, HLAE, ULBP1, ULBP256, and ULBP3. In another embodiment said tumor cells express MICA, MICB, HLAE, ULBP1, ULBP256, and ULBP3. In other embodiments, as exemplified herein, the cell compositions are capable of specifically eradicating said hematopoietic tumor cells to a greater extent than CIK compositions under the same conditions (e.g. 6-10-fold higher at a 1:1 effector cell to tumor cell ratio or up to about 15-fold higher at a 0.5:1 effector cell to tumor cell ratio). In other embodiments, said tumor cells are solid tumor cells. In a particular embodiment, said cells are prostate tumor cells or mesothelioma cells. Each possibility represents a separate embodiment of the invention.

As used herein, the term “specific eradication” with respect to tumor cells indicates targeted tumor cell death, which is selective (or preferential) to the target tumor cells. Typically, eradication is exerted by cytotoxic activity that is induced by said tumor cells (e.g. upon contact of the cell composition with said tumor cells under conditions as disclosed herein).

As used herein, the terms “cytotoxicity” and “cytotoxic activity” refer in particular to cell-mediated cytotoxicity or cytolysis, i.e. to the cell killing activity of immune cells, in particular of effector immune cells. Among the prominent cytotoxic effector cells of the immune system are NK cells, cytotoxic T cells (e.g., CD8⁺ T cells) and NK-T cells. A cytotoxic cell (e.g. CTL or NK cell) may kill a target cell via target cell apoptosis or lysis using one or more different mechanisms including, but not limited to, release of one or more cytotoxins stored in cytolytic granules, or expression of a Fas ligand (FasL). Exemplary cytotoxins include e.g. a perforin, a granzyme and a granulysin. Currently known granzymes are Granzymes A, B, H, K and M. Such cytotoxic or cytolytic activities can be measured and quantified using standard techniques, e.g., by assays employing selective labeling of viable cells. For example, without limitation, diacetate succinimidyl ester (CFSE)- and/or propidium iodide-based assays, or other suitable assays known in the art may be employed.

In another embodiment, the cytotoxic activity is mediated by Granzyme B and/or perforin. In another embodiment said cytotoxic activity is not substantially mediated by FAS-FasL interactions.

Perforin is a pore forming cytolytic protein which translocate to the target cell in a Ca⁺²-dependent manner. Upon release of cytolytic granules in close proximity to a cell slated for killing, perforin forms pores in the cell membrane of the target cell through which the granzymes and associated molecules can enter, inducing apoptosis. Granzyme B (also known as granzyme 2 and cytotoxic T-lymphocyte-associated serine esterase 1), is a particularly important serine protease eliciting rapid induction of target cell apoptosis in the cell-mediated immune response, by cleavage and activation of caspases. The FAS receptor, also known as Fas, FasR, apoptosis antigen 1 (APO-1 or APT), cluster of differentiation 95 (CD95) or tumor necrosis factor receptor superfamily member 6 (TNFRSF6), is a death receptor on the surface of cells that leads to programmed cell death (apoptosis) upon binding its ligand, Fas ligand (FasL). Expression of FasL by immune effector cells and subsequent FAS-FasL interactions upon contact with FAS-expressing target tumor cells may induce tumor cell apoptosis in a perforin-independent manner. Such perforin-independent cytotoxic activities may be examined in Ca2+-independent systems in which the perforin monomer is unable to polymerize but cell-mediated cytolysis still occurs.

As used herein, a cytotoxic activity mediated by a specific agent or pathway (e.g. perforin, Granzyme B or FAS-FasL) refers to an activity that is triggered by and/or is substantially dependent on the involvement or activity said agent. Such activity requires that said agent be expressed and subsequently activated in the corresponding effector cells. Thus, a significant reduction in cytotoxic activity in the presence of a specific pharmacological inhibitor or antibody directed to the agent or pathway (typically at least 50% and more typically at least 60%, 70%, 80%, 90% and up to 100% inhibition) compared to the activity under the same conditions in the absence of said inhibitor or antibody, indicates that the activity is mediated by the agent (e.g. perforin or Granzyme B). Similarly, lack of significant reduction (statistically significant, or in some embodiments no more than 5%, 10%, 20% or 30% reduction) indicates that the activity is not mediated by the agent (e.g. FasL). For example, cytotoxicity assays as disclosed herein may be performed in the presence of 3,4-Dichloroisocoumarin (DCI) or other commercially available granzyme B inhibitors, Concanamycin A (CMA) or other commercially available perforin inhibitors, or anti-FasL (or anti-FAS) blocking antibodies commercially available from Sigma or other vendors. Non-limitative examples of such assays are provided in the Examples section below.

As demonstrated herein, cell compositions in accordance with the invention were found to possess both MHC-restricted and non-MHC restricted cytotoxic activities against various tumor cells. Thus, in one embodiment, the cytotoxic activity is MHC-dependent (i.e. requires the presence or activity of MHC molecules on the cells). In another embodiment, the cytotoxic activity is MHC-independent. In another embodiment, the cytotoxic activity is substantially MHC I-independent. In another embodiment, the cytotoxic activity is substantially MHC II-independent.

In another embodiment, the cell compositions of the invention are capable of exerting tumor-specific cytotoxic activity without substantial cytotoxic activity against non-tumor cells. In other embodiments, the cell compositions of the invention do not substantially induce graft-versus-host (GVH) reaction or graft-versus-host disease (GVHD). Thus, in some embodiments, the cell compositions of the invention need not be histocompatible with the subject to be treated. Accordingly, autologous, histocompatible allogeneic, and non-histocompatible allogeneic PBMC samples may be used in the preparation of the compositions of the invention.

term “graft-versus-host disease” or “GVHD” as used herein refers to an inflammatory disease resulting from an attack by transplanted leukocytes against the tissues of the transplant recipient. GVHD typically occurs in an immunocompromised host when it is recognized as non-self by immunocompetent cells of a graft, and encompasses acute and/or chronic GVHD. In the context of ACT, the term GVHD refers in particular to the pathological process and clinical manifestation resulting from damage to non-tumor cells, tissues and/or organs of the recipient subject, induced by the adoptively transferred immune cells. GVHD is often manifested as inflammation-mediated damage to epithelial tissues, especially skin, liver, and mucosa of the gastrointestinal tract.

The term “histocompatibility” refers to the similarity of tissue between different individuals. The level of histocompatibility describes how well matched the patient and donor are. The major histocompatibility determinants are the human leukocyte antigens (HLA). HLA typing is performed between the potential donor and the potential recipient to determine how close a HLA match the two are. The term “histocompatible” as used herein refers to embodiments in which all six of the HLA antigens (2 A antigens, 2 B antigens and 2 DR antigens) are the same between the donor and the recipient.

As further demonstrated herein, the cell compositions of the invention exhibit significant cytotoxic activity against tumor cells (including against non-histocompatible tumor cells) in vivo. For example, as exemplified in Example 8 herein, compositions in accordance with the invention exhibited a remarkable cytotoxic activity against lymphoid tumor cells, inhibiting tumor development by at least 78%-97%. Accordingly, the invention relates in some embodiments to cell compositions capable of inhibiting the development of hematopoietic tumors in the spleen by at least 50%, at least 60%, at least 70% or at least 80%, 90%, 95% or 97% wherein each possibility represents a separate embodiment of the invention. Further, advantageous compositions in accordance with the invention compositions of the invention exhibit cytotoxic activity against non-histocompatible tumor cells in vivo that is significantly higher than that exerted by PBMC (which are generally considered not substantially effective in eradicating tumor cells in vivo) or various PBMC-derived compositions such as CIK and LAK.

In another embodiment, there is provided an ACT cell composition comprising 5×10⁶ to 10×10⁹ viable in vitro-expanded PBMC, of which 70-85% are CD3⁺CD8⁺ cells expressing NKG2D, DNAM-1⁺ and granzyme B, at least 65% are CD56⁻ cells, and up to 8% are CD3⁻ cells, said composition exhibiting significant cytotoxic activity against tumor cells in vitro and in vivo. In another embodiment, 5-15% of the viable cells are CD3⁺CD4⁺ cells, 15-30% are CD3⁺CD56⁺ cells, and 7-17% are CD3⁺CD8⁺CD56⁺ cells. In another embodiment, the cell composition is characterized by a ratio of CD3⁺CD8⁺ cells to CD3⁺CD4⁺ cells of 7-8, and/or comprise less than 5% CD3⁻CD56⁺ cells.

### Genetically Modified Cells

In another embodiment, the cell compositions of the invention are genetically modified. In one embodiment, the cells have been modified to express at least one receptor providing tumor specificity, e.g. a recombinant T cell receptor (TCR) or a chimeric antigen receptor (CAR). In another embodiment, there is provided a cell composition comprising a recombinant construct of the invention, or a unique combination of recombinant constructs as disclosed hereinbelow.

A CAR combines the binding site of a molecule that attaches strongly to the antigen being targeted (i.e., a “binding portion”) with the cytoplasmic domains of conventional immune receptors responsible for initiating signal transduction that leads to lymphocyte activation (the “signaling portion”). Most commonly, the binding portion used is derived from the structure of the Fab (antigen binding) fragment of a monoclonal antibody (mAb) that has high affinity for the antigen being targeted. Because the Fab is the product of two genes, the corresponding sequences are usually combined via a short linker fragment that allows the heavy-chain to fold over the light-chain derived peptides into their native configuration, creating a single-chain fragment variable (scFv) region. As many known as the original CARs systems attached an antibody fragment to a T cell, they were also called “T-bodies”. Other possible antigen binding moieties include signaling portions of hormone or cytokine molecules, the extracellular domains of membrane receptors and peptides derived from screening of libraries (e.g. phage display). Suitable antigenic targets for CAR used in the compositions of the invention are disease specific antigens as disclosed herein.

The term “antibody” is meant to include both intact molecules as well as fragments thereof that include the antigen-binding site. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1). Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single-chain Fvs is provided in the literature of the art.

With respect to the cytoplasmic domain, the CAR can be designed to comprise signaling domains of co-stimulatory molecules, e.g. the CD80, CD86, CD40, CD83, 4-1BB (CD137), CD28 and/or CD3ζ signaling domain by itself or combined with any other desired cytoplasmic domain(s) useful in the context of the CAR. In another embodiment, the CAR comprises a signaling domain of a co-stimulatory molecule selected from the group consisting of 4-1BB, CD28, CD3ζ, or a combination thereof. In one exemplary embodiment, the CAR-modified cells of the invention can be generated by introducing a viral vector such as a lentiviral vector comprising a desired CAR, for example a CAR comprising anti-CD19 binding domain, a transmembrane domain, and a cytoplasmic signaling domain, into the cells. Alternatively, a vector is used that is stably maintained in the cell, without being integrated in its genome. In another embodiment, the CAR-modified cells of the invention can be generated by transduction or transfection of a gene encoding such a CAR molecule in the cell.

In a particular embodiment said cells are genetically modified to express a CAR, e.g. a CD19-specific CAR or a CD123-specific CAR. B-lymphocyte antigen CD19, also known as CD19 molecule (Cluster of Differentiation 19), B-Lymphocyte Surface Antigen B4, T-Cell Surface Antigen Leu-12 and CVID3 is a transmembrane protein that in humans is encoded by the gene CD19. In humans, CD19 is expressed in all B lineage cells. CD19 plays two major roles in human B cells: on the one hand, it acts as an adaptor protein to recruit cytoplasmic signaling proteins to the membrane; on the other, it works within the CD19/CD21 complex to decrease the threshold for B cell receptor signaling pathways. Due to its presence on all B cells, it is a biomarker for B lymphocyte development, lymphoma diagnosis and can be utilized as a target for leukemia immunotherapies. The interleukin-3 receptor (CD123) is a molecule found on cells which helps transmit the signal of interleukin-3, a soluble cytokine important in the immune system. The gene coding for the receptor is located in the pseudoautosomal region of the X and Y chromosomes. The receptor belongs to the type I cytokine receptor family and is a heterodimer with a unique alpha chain paired with the common beta (beta c or CD131) subunit. The gene for the alpha subunit is 40 kilobases long and has 12 exons.

In certain exemplary embodiments, the CAR comprises scFv of an anti-CD19 antibody linked to 4-1BB and CD3ζ signaling domains. In another exemplary embodiment, said CAR may further comprise additional domains, including, but not limited to CD8-derived hinge and/or transmembrane regions. In other exemplary embodiments, the CAR may comprise a scFv of anti-CD19 antibody linked to CD28 and CD3ζ signaling domains. Non-limitative examples of specific CAR molecules and constructs encoding same that can be used in accordance with embodiments of the invention are described in Examples 8 and 9 below.

In other embodiments, CAR directed to other targets, e.g. BCMA, CD47, PDL-1, mesothelin, EpCAM, CD34, CD44, PSCA, MUC16, CD276, CD123, CD19, CD20 and EFFRvIII may be used. In yet other embodiments, various other markers may be employed as CAR targets, including, but not limited to, AFP, Axl, B7-H6, BCMA, Biotin, CD10, CD117, CD123, CD133, CD138, CD19, CD20, CD200, CD22, CD276, CD30, CD33, CD34, CD38, CD44, CD5, CD70, CD79A, CD80, CD86, CD99, CEA, CLDN18.2, c-Met, CS1, DLL3, DR5, EGFR, EGFRvIII, EpCAM, EphA2, ErbB2, ERBB3, FAP, Fibroblast activation protein (FAP), FRα, GD2, GPC3, IL1RAP, ITGB7, LeY, mesothelin, MUC1, MUC16, PD-1, PD-L1, PMEL, PSCA, PSMA, and VEGFR2. Sequences of various receptors are available and may be used to generate nucleic acid constructs for genetic modification. For example, without limitation, CAR constructs directed to CD19 or CD123, used to modify T cell compositions, are disclosed in US20140271635 and US20140322212, respectively.

In other embodiments, a construct encoding a recombinant TCR, directed against e.g. a tumor associated antigen (TAA) or other targets useful in the treatment of cancer, may be used. Non-limitative examples of well-known TAA are MART-1, gp100₂₀₉₋₂₁₇, gp100₁₅₄₋₁₆₃, CSPG4, NY-ESO, MAGE-A1, and Tyrosinase. Such TCR constructs are known in the art and may be obtained or generated by recombinant methods.

In another embodiment the cells have been modified to express at least one cytokine, chemokine, or a receptor thereof. In one embodiment, the cells are genetically modified to express a modified chemokine receptor. In one embodiment, the chemokine receptor has been modified to enhance tumor localization or retention. As used herein, enhanced tumor localization or retention relates to the ability of the adoptively transferred effector cells to migrate, penetrate and/or proliferate at the tumor microenvironment, and is manifested as enhancement in the relative number of effector cells that may be identified in the tumor following adoptive transfer compared to their levels in the absence of such transgenic receptor. For example (e.g. when the tumor to be treated is derived from, or resides in, the bone marrow), the modified chemokine receptor advantageously enhances bone marrow retention of the transgenic cells. In a particular embodiment, the modified chemokine receptor is a modified CXCR4 receptor comprising a mutation or truncation at the C-terminal tail domain (resulting in e.g. enhanced tumor localization and/or retention as described herein). For example, WHIM mutations at the CXCR4 gene (identified in patients of WHIM syndrome) are associated with C-terminal truncation of the encoded receptor, e.g. removal of 15-20 C-terminal amino acids (aa). In a particular embodiment, said modified CXCR4 is characterized by a 19 C-terminal aa truncation, e.g. due to the R334X mutation (also referred to herein as WHIM R334x mutation, CXCR4 containing a truncation mutation after residue 333, exemplified by SEQ ID NO: 8 as set forth below). By means of non-limitative examples, such modified cells are particularly useful in treating hematopoietic tumors and metastases localized to the bone marrow.

In another embodiment, the cells have been genetically modified to express at least one cytokine of the IL-2 family. For example, the cells may be modified to express exogenously (e.g. in a constitutive or otherwise up-regulated manner) IL-2, IL-15 and/or IL-21. Each possibility represents a separate embodiment of the invention. In another embodiment, the cell composition expresses any one of exogenous IL-2, exogenous IL-15, exogenous IL-21, or any combination thereof. The term “exogenous,” when used in relation to a protein, gene, nucleic acid, or polynucleotide in a cell or organism refers to a protein, gene, nucleic acid, or polynucleotide that has been introduced into the cell or organism by artificial or natural means. An exogenous nucleic acid may be from a different organism or cell, or it may be one or more additional copies of a nucleic acid that occurs naturally within the organism or cell. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

IL-15 is a cytokine with structural and functional similarity to IL-2, discussed above. Like IL-2, IL-15 binds to and signals through a complex composed of IL-2/IL-15 receptor beta chain (CD122) and the common gamma chain (gamma-C, CD132). IL-15 is encoded by the IL15 gene and induces the proliferation of NK cells. IL-21 is encoded by the IL21 gene in humans, and induces proliferation of the immune system, including NK cells and cytotoxic T cells.

In some embodiments, the cells are characterized by a plurality of modifications. According to a non-limitative advantageous embodiment, disclosed herein are cell compositions modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. As disclosed herein, in some embodiments such modified SAK cell compositions are particularly useful for the treatment of hematopoietic tumors and tumors localized to the bone marrow such as leukemias. In some embodiments, the cells have been modified by transfection, transduction or infection with a nucleic acid construct encoding: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. In another embodiment the construct is a viral vector. In another embodiment the construct further comprises at least one translation modulating sequence e.g. an internal ribosome entry site (IRES) sequence or a self-cleaving sequence such as a 2A peptide sequence. In an exemplary embodiment, the translation modulating sequence is located between the nucleic acid sequences encoding elements (i) and (ii) and/or between elements (ii) and (iii). In another particular embodiment said elements are constitutively expressed.

In another embodiment there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21.

In another embodiment, there is provided a nucleic acid construct encoding: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. In yet another embodiment, there is provided a nucleic acid construct of the invention as disclosed herein.

The preparation of nucleic acid constructs, including in particular expression constructs or vectors used for delivering and expressing a desired gene product are known in the art. An isolated nucleic acid sequence can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis (see e.g. Sambrook et al., 2001, Molecular Cloning: A Laboratory Manual, Cold Springs Harbor Laboratory, New York; Ausubel, et al., 1989, Chapters 2 and 4). Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional peptide. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide or polypeptide analog and the polypeptide is synthesized in the host upon transcription and translation of the DNA sequence.

Exemplary sequences of cytokines, chemokine receptors, CAR and other genetic elements that may be cloned into constructs in accordance with embodiments of the invention are provided in Examples 8 and 9 below. For instance, an exemplary nucleic acid sequence of human IL-2 is denoted by SEQ ID NO: 3, an exemplary nucleic acid sequence of human IL-15 is denoted by SEQ ID NO: 4, an exemplary nucleic acid sequence of wild-type CXCR4 is denoted by SEQ ID NO: 7, an exemplary nucleic acid sequence of a modified, C′-truncated CXCR4 is denoted by SEQ ID NO: 8, an exemplary nucleic acid sequence of a CD19-specific CAR is denoted by SEQ ID NO: 9, and an exemplary nucleic acid sequence of a CD123-specific CAR is denoted by SEQ ID NO: 1. In other embodiments, functionally equivalent variants and homologs of these sequences, retaining at least 90% sequence identity, e.g. 93%, 95%, 97%, or 99% homology, may be used.

The construct may also comprise other regulatory sequences or selectable markers, as known in the art. The nucleic acid construct (also referred to herein as an “expression vector”) may include additional sequences that render this vector suitable for replication and integration in prokaryotes, eukaryotes, or optionally both (e.g., shuttle vectors). In addition, a typical cloning vector may also contain transcription and translation initiation sequences, transcription and translation terminators, and a polyadenylation signal.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives. These may serve as vector backbone for the constructs useful in embodiments described herein.

Expression vectors containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Barr virus include pHEBO and p2O5. Other exemplary vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells. These may serve as vector backbone for the constructs of the present invention.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinarily skilled artisan and as such, no general description of selection considerations is provided herein.

Retroviral-derived vectors include e.g. lentiviral vectors. “Lentiviral vector” and “recombinant lentiviral vector” are derived from the subset of retroviral vectors known as lentiviruses. Lentiviral vectors refer to a nucleic acid construct which carries, and within certain embodiments, is capable of directing the expression of a nucleic acid molecule of interest. The lentiviral vector includes at least one transcriptional promoter/enhancer or locus defining element(s), or other elements which control gene expression by other means such as alternate splicing, nuclear RNA export, post-translational modification of messenger, or post-transcriptional modification of protein. Such vector constructs must also include a packaging signal, long terminal repeats (LTRS) or portion thereof, and positive and negative strand primer binding sites appropriate to the lentiviral vector used (if these are not already present in the retroviral vector). Optionally, the recombinant lentiviral vector may also include a signal which directs polyadenylation, selectable markers such as Neo, TK, hygromycin, phleomycin, histidinol, or DHFR, as well as one or more restriction sites and a translation termination sequence. By way of example, such vectors typically include a 5′ LTR, a tRNA binding site, a packaging signal, an origin of second strand DNA synthesis, and a 3′LTR or a portion thereof.

“Lentiviral vector particle” may be utilized within the present invention and refers to a lentivirus which carries at least one gene of interest. The retrovirus may also contain a selectable marker. The recombinant lentivirus is capable of reverse transcribing its genetic material (RNA) into DNA and incorporating this genetic material into a host cell's DNA upon infection. Lentiviral vector particles may have a lentiviral envelope, a non-lentiviral envelope (e.g., an amphotropic or VSV-G envelope), or a chimeric envelope.

In some embodiments, the modified cell composition is a SAK cell composition or an ACT cell composition of the invention as disclosed herein. For example, an ACT cell composition comprising at least 5×10⁶ to 10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, may be subjected to the genetic modifications as disclosed herein.

In other embodiments, other leukocyte preparations or populations, including, but not limited to, T cells, NK cells, NK-T cells and DC, as well as various ACT compositions known in the art (e.g. TIL, CIK and LAK), may be used for modification. However, it should be understood that the use of such leukocytes that have not been modified by the improved constructs of the invention as disclosed herein is to be distinguished from the teachings and principles of the invention. According to particular embodiments, the use of such alternative leukocyte compositions is explicitly excluded.

### Therapeutic Use

In another aspect, the cell composition of the invention may be used in the treatment of cancer. In another aspect, there is provided a method of treating a tumor in a subject in need thereof, comprising contacting the tumor cells with a therapeutically effective amount of a cell composition of the invention, thereby treating the tumor in the subject. In another embodiment, there is provided an ACT composition, comprising at least 5×10⁶-10×10⁸ viable in vitro-expanded peripheral blood mononuclear cells (PBMC), of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo, for use in treating a tumor in a subject in need thereof. In another embodiment, there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21, for use in treating a tumor in a subject in need thereof.

As used herein, the terms “tumor” and “cancer” may be used synonymously and refer to both solid tumors and non-solid malignancies. In another embodiment, the tumor is a hematopoietic tumor. Non-limitative examples of hematopoietic tumors of lymphoid lineage include e.g. leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkitt's lymphoma. Non-limitative examples of hematopoietic tumors of myeloid lineage, include e.g. acute and chronic myelogenous leukemias, myelodysplastic syndrome and promyelocytic leukemia; tumors of mesenchymal origin, including fibrosarcoma and rhabdomyosarcoma. In another embodiment the tumor is a solid tumor. Non-limitative examples of solid tumors include e.g. lung tumors, stomach tumors, ovarian tumors, breast tumors, colorectal tumors, breast tumors, pancreatic tumors, and renal tumors. In various embodiments, said tumor is selected from the group consisting of leukemia, multiple myeloma, prostate cancer mesothelioma, lung cancer and pancreatic cancer, wherein each possibility represents a separate embodiment of the invention. In a specific embodiment, said tumor is selected from the group consisting of leukemia, multiple myeloma, prostate cancer and mesothelioma. In a particular embodiment said tumor is AML.

In another embodiment said tumor is characterized by expression of at least one NKG2D ligand. In some embodiments, the NKG2D ligand is selected from the group consisting of MICA, MICB, HLAE, ULBP1, ULBP256, and ULBP3. In another embodiment said tumor expresses a plurality of NKG2D ligands selected from the group consisting of: MICA, MICB, HLAE, ULBP1, ULBP256, and ULBP3. In another embodiment said tumor expresses MICA, MICB, HLAE, ULBP1, ULBP256, and ULBP3. Each possibility represents a separate embodiment of the invention.

Conveniently, the expression of markers such as NKG2D ligands and checkpoint molecule ligands by a tumor of a subject may be determined by obtaining tumor cells (e.g. from a solid tumor biopsy or from bodily fluids such as blood or urine), and examining expression of said markers on the surface of said tumor cells by suitable assays including, but not limited to immunostaining and flow cytometry. Non-limitative examples of such assays are exemplified herein.

In another embodiment, said tumor is characterized by expression of at least one inhibitory immune checkpoint molecule and/or ligand thereof. In some embodiments, said tumor may express at least one ligand of CTLA-4, PD-1, TIGIT, Lag-3, Tim-3 and combinations thereof, wherein each possibility represents a separate embodiment of the invention. In another embodiment, said tumor is characterized by expression of at least one ligand of an inhibitory immune checkpoint molecule selected from the group consisting of CTLA-4, PD-1, and TIGIT. In another embodiment, said tumor is characterized by expression of ligands of a plurality of inhibitory immune checkpoint molecule selected from the group consisting of CTLA-4, PD-1, and TIGIT. In another embodiment, said tumor is characterized by expression of at least one CTLA-4 ligand (e.g. CD80 and/or CD86).

In another embodiment, said tumor is resistant to treatment by at least one checkpoint molecule inhibitor. In another embodiment, said tumor is resistant to treatment by at least one inhibitor (e.g. blocking antibody) directed to at least one checkpoint molecule selected from the group consisting of CTLA-4, PD-1, TIGIT, Lag-3, Tim-3 and ligands and combinations thereof, wherein each possibility represents a separate embodiment of the invention.

According to exemplary embodiments, the tumor is resistant to treatment by at least one CTLA4-specific inhibitor (e.g. blocking antibody) selected from the group consisting of ipilimumab (YERVOY®) and tremelimumab. In other embodiments, the tumor is resistant to treatment by at least one PD1-specific inhibitor (e.g. blocking antibody directed to PD-1 or PD-L1) selected from the group consisting of atezolizumab (TECENTRIQ®), durvalumab (IMFINZI®), avelumab (BAVENCIO®), nivolumab (OPDIVO®) and pembrolizumab (KEYTRUDA®). In other embodiments, the tumor is resistant to treatment by at least one TIGIT-specific inhibitor selected from the group consisting of tiragolumab (MTIG7192A), ociperlimab (BGB-A1217), vibostolimab (MK-7684), domvanalimab (AB-154), BMS-986207, EOS-448, ASP-8374, COM-902, etigilimab (MPH-313), IBI-939, AGEN-1307, CASC-674, Anti-PVR antibody (NB-6253), and PH-804. In other embodiments, the tumor is resistant to treatment by at least one LAG3-specific inhibitor selected from the group consisting of PRS-332, P13B02-3, LBL-007, eftilagimod alpha (IMP321), LAG525 (IMP701), MK-4280, REGN3767, relatlimab (BMS-986016), BI 754111, FS118, tebotelimab (MGD013), TSR-033, INCAGN2385, Sym022 and XmAb22841. In other embodiments, the tumor is resistant to treatment by at least one TIM3-specific inhibitor, e.g. ICAGN02390, Sym023, LY3321367, MGB453, TSR022, BGBA425, and BMS986258.

In another embodiment, the tumor is resistant to treatment by a plurality of checkpoint inhibitors or by inhibitors of a plurality of checkpoint molecules. For example, without limitation, the tumor may be resistant to treatment by a dual inhibitor targeting both TIM3 and PD1 such as R07121661 or to combination therapy with nivolumab and ipilimumab.

In another embodiment, said subject is under treatment regimen with one or more checkpoint molecule inhibitors. In another embodiment, said subject is not under treatment regimen with checkpoint molecule inhibitors. For example, YERVOY™ (ipilimumab) is a human CTLA-4-blocking antibody for intravenous infusion, indicated for the treatment of unresectable or metastatic melanoma. OPDIVO® (nivolumab) is a PD-1-blocking antibody indicated for the treatment of unresectable or metastatic melanoma. KEYTRUDA® (pembrolizumab) is a PD-1-blocking antibody indicated for treatment of melanoma, NSCLC, head and neck squamous cell cancer, classical hodgkin lymphoma (cHL), primary mediastinal large B-cell lymphoma, urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden-high cancer, cutaneous squamous cell carcinoma, and triple-negative breast cancer. TECENTRIQ® (atezolizumab) is a programmed death-ligand 1 (PD-L1) blocking antibody indicated for the treatment of locally advanced or metastatic urothelial carcinoma, metastatic NSCLC, metastatic non-squamous NSCLC with no EGFR or ALK genomic tumor aberrations, Small Cell Lung Cancer (SCLC), extensive-stage small cell lung cancer (ES-SCLC), unresectable or metastatic HCC, and melanoma. IMFINZI® (durvalumab) is a PD-L1 blocking antibody indicated for the treatment of adult patients with unresectable, Stage III NSCLC whose disease has not progressed following concurrent platinum-based chemotherapy and radiation therapy; in combination with etoposide and either carboplatin or cisplatin, as first-line treatment of adult patients with ES-SCLC. BAVENCIO® (avelumab) is a PD-L1 blocking antibody indicated for the treatment of MCC, UC, and RCC.

In another embodiment, the method or use further comprises administration of at least one checkpoint molecule inhibitor as disclosed herein (in concurrent or sequential combination with the cell composition of the invention). For example (e.g. wherein the tumor is characterized by surface expression of at least one ligand of Lag-3, Tim-3 or TIGIT), the method of use further comprises administration of at least one checkpoint molecule inhibitor directed to Lag-3, Tim-3 or TIGIT, respectively. In another embodiment, the method or use further comprises administration of at least one checkpoint molecule inhibitor directed to Lag-3, Tim-3, or combinations thereof. Yet in other embodiments, cell compositions of the invention may be used as the sole therapeutic agent. For example, without limitation, compositions of the invention may be used as monotherapy in the treatment of tumors characterized by surface expression of at least one ligand of CTLA-4 or PD-1. Each possibility represents a separate embodiment of the invention.

In another embodiment, said tumor is characterized by expression of at least one chemokine selected from the group consisting of CXCR6 ligands, CXCR4 ligands, CXCR2 ligands and CXCR3 ligands. In another embodiment, said tumor is characterized by expression of at least one chemokine selected from the group consisting of CXCR6 ligands, CXCR4 ligands, and CXCR3 ligands. In another embodiment, said tumor is characterized by expression of a plurality of chemokines selected from the group consisting of CXCR6 ligands, CXCR4 ligands, CXCR2 ligands and CXCR3 ligands. In another embodiment, said tumor is characterized by expression of a plurality of chemokines selected from the group consisting of CXCR6 ligands, CXCR4 ligands, and CXCR3 ligands. In a particular embodiment said tumor is characterized by expression of a CXCR4 ligand, e.g. CXCL12. In another particular embodiment said tumor is characterized by expression of a CXCR6 ligand, e.g. CXCL16. For example, without limitation, CXCL16-expressing tumors may include non-small cell lung cancer (NSCLC) tumors, stomach tumors, ovarian tumors, breast tumors, colorectal tumors, breast tumors, pancreatic tumors, and renal tumors, wherein each possibility represents a separate embodiment of the invention.

In another embodiment the contacting is performed in vivo. Thus, the methods of the invention may comprise administering to said subject a therapeutically effective amount of a cell composition of the invention, e.g. an ACT cell composition, comprising at least 5×10⁶-10×10⁸ viable in vitro-expanded PBMC, of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo. In another embodiment the contacting is performed ex vivo. In another embodiment, the cells are administered to said subject with concomitant IL-2 administration. Yet in other embodiments (e.g. when the improved genetically modified cells are used), the cells may advantageously be used in the absence of concomitant IL-2 administration, thereby providing enhanced safety and reduced side effects.

An “effective amount” or “therapeutically effective amount” refers to an amount sufficient to exert a beneficial outcome in a method of the invention. More specifically, a therapeutically effective amount means an amount of active ingredients (e.g., effector cells) effective to prevent, alleviate, or ameliorate symptoms of a disorder (e.g., cancer) or prolong the survival of the subject being treated. For example, an ACT composition of the invention for administration to a subject comprises an effective amount of at least 5×10⁶-10×10⁸ and typically up to about 10×10⁹ viable cells expanded from PBMC as disclosed herein. an effective amount of a cell composition for ex vivo contacting with tumor cells may comprise for example at least about 0.25-1 effector cells for every tumor cell in culture.

In the context of in vitro cell culture methods, an effective amount of cell stimulators such as IL-2 and CD3 activators is an amount sufficient to exert advantageous phenotypic modulations as disclosed herein, including, but not limited to cell expansion, up-regulation of activation markers (e.g. DNAM-1, NKG2D) and enhancement in cytotoxic capacity. For example, without limitation, effective amounts for IL-2 may include e.g. 500-1500, 600-1300, 750-1500 or 500-1200 IU/ml for recombinant human IL-2, and for anti-CD3 antibodies e.g. 10-60, 20-40, 10-40 or 20-60 ng/ml for OKT3.

Determination of a therapeutically effective amount is well within the capability of those skilled in the art, especially in light of the detailed disclosure provided herein. For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p.1.).

In another embodiment, said tumor is characterized by down-regulation of MHC I expression and/or activity. As demonstrated herein, cell compositions in accordance with the invention were found to possess both MHC-dependent and non-MHC dependent cytotoxic activities against various tumor cells. Accordingly, these cell compositions may advantageously be used even in the treatment of treatment-resistant tumors (e.g. refractory to immunotherapy or checkpoint molecule inhibitors) or tumors that exhibit an immune-evasive phenotype associated with impaired MHC I antigen presentation due to e.g. reduced expression of MHC I molecules, loss of function mutations or other impairments in the MHC I pathway characteristic of tumors.

In another embodiment, said cell composition had undergone cryopreservation and a subsequent thawing protocol prior to contacting with said tumor cells. As exemplified herein, cell compositions in accordance with the principles of the invention exhibited highly potent cytotoxicity following cryopreservation and thawing. Further, this activity was manifested even close to the time of thawing (retaining 70-80% of the cytotoxic activity of corresponding non-cryopreserved cells, within 24 hours of thawing), without requiring additional steps of expansion or manipulation. Thus, in another embodiment of the methods of the invention, the thawing protocol is performed within 1-3 days, e.g. within 36, 24, 12 or 6 hours of contacting said cell composition with said tumor cells. Each possibility represents a separate embodiment of the invention. In other embodiments, the cell composition may be autologous, histocompatible allogeneic, or non-histocompatible allogeneic to said subject, wherein each possibility represents a separate embodiment of the invention.

### Additional Embodiments

In one aspect, the invention provides a process for producing a cell composition for ACT, comprising:
a. providing a PBMC sample,
b. expanding the PBMC in the constant presence of effective amounts of IL-2 and a CD3 activator for at least 9 days, and in the absence of other cytokines or antibodies, wherein effective amounts of the IL-2 and CD3 activator are supplemented every 2-4 days, so as to enhance the number of viable cells in the culture by at least 20-fold, to thereby obtain a cell composition comprising 5×10⁶ to 10×10⁸ viable cells, and
c. collecting the resulting cell composition.

In another embodiment, expansion is performed in the constant presence of said IL-2 and CD3 activator as sole exogenously-added cell stimulators. In a particular embodiment, step b. comprises expanding the cells in the presence of 500-1500 IU/ml IL-2 and 10-60 ng/ml anti-CD3 antibody, which are re-supplemented every 2-4 days, for 10-16 days. In another embodiment the cells have not been subjected to additional enrichment, stimulation or expansion steps. In another embodiment the expansion is performed so as to enhance the number of viable cells by at least 30-fold. In another embodiment expansion is performed so as to produce a cell composition comprising 5×10⁶ to 10×10⁸ viable mononuclear cells, of which 70-80% are CD3⁺CD8⁺ cells expressing NKG2D and granzyme B. In another embodiment, expansion is performed so as to produce a cell composition capable of specifically eradicating hematopoietic tumor cells in a significant manner when incubated with the tumor cells in vitro at a ratio of 0.25:1 for 24 hours. In another embodiment, expansion is performed so as to produce a cell composition of the invention, as disclosed herein.

In another embodiment step b. further comprises genetically modifying the cells. In another embodiment, the process comprises genetically modifying said cells to express a CAR and/or at least one cytokine, chemokine, or a receptor thereof. In an exemplary embodiment, the process comprises modifying the cells to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. In another embodiment, the method further comprises a step of cryopreserving the cell composition collected at step c.

In another embodiment, there is provided a cell composition prepared by a process as disclosed herein.

In another aspect, there is provided an ACT cell composition, comprising 5×10⁶ to 10×10⁸ viable in vitro-expanded PBMC, of which 70-80% are CD3⁺CD8⁺ cells expressing NKG2D and granzyme B, at least 70% are CD56⁻ cells, and up to 5% are CD3⁻ cells, said composition exhibiting significant cytotoxic activity against non-histocompatible tumor cells in vitro and in vivo.

In one embodiment, 7-12% of the viable cells are CD3⁺CD4⁺ cells, 20-28% are CD3⁺CD56⁺ cells, and 10-14% are CD3⁺CD8⁺CD56⁺ cells. In another embodiment, the composition is characterized by a ratio of CD3⁺CD8⁺ cells to CD3⁺CD4⁺ cells of 7-8. In another (additional or alternative) embodiment, the composition comprises less than 3% CD3⁻CD56⁺ cells.

According to certain embodiments, at least 40% of the CD3⁺CD8⁺ cells are characterized by surface expression of at least one marker selected from the group consisting of perforin, FasL, CXCR4 and CXCR2. In another embodiment, at least 40% of the CD3⁺CD8⁺ cells are characterized by surface expression of a plurality of markers selected from the group consisting of perforin, FasL, CXCR4 and CXCR2. In some embodiments, about 40% of the CD3⁺CD8⁺ cells express FasL, about 50% of the CD3⁺CD8⁺ cells express perforin, about 90% of the CD3⁺CD8⁺ cells express CXCR2, and about 100% of the CD3⁺CD8⁺ cells express CXCR4, Granzyme B and NKG2D.

In another embodiment, at least 30% and typically at least about 40% of the CD3⁺CD8⁺ cells are characterized by surface expression of at least one marker selected from the group consisting of CXCR3, CXCR6 and DNAM-1. In one embodiment, at least 90% of the CD3⁺CD8⁺cells are characterized by surface expression of CXCR3. In another embodiment, at least 90% and typically substantially all of the CD3⁺CD8⁺ cells are characterized by surface expression of DNAM-1. In another embodiment, at least 30% of the CD3⁺CD8⁺ cells are characterized by surface expression of CXCR6. In another embodiment, about 90% of the CD3⁺CD8⁺ cells are characterized by surface expression of CXCR3, substantially all of the CD3⁺CD8⁺ cells are characterized by surface expression of DNAM-1, and about 30% of the CD3⁺CD8⁺ cells are characterized by surface expression of CXCR6. Thus, ACT cell compositions according to embodiments of the invention comprise a CD3⁺CD8⁺ cell population defined as NKG2D⁺, granzyme B⁺, perforin*, FasL*, CXCR4⁺, CXCR2⁺, CXCR3⁺, CXCR6⁺ and DNAM-1⁺.

In another embodiment, no more than 55% and typically up to about 50% of the CD3⁺CD8⁺ cells are characterized by surface expression of one or more immune checkpoint molecules selected from the group consisting of CTLA-4, PD-1, TIGIT, Lag-3 and Tim-3. In another embodiment, no more than about 50% of the CD3⁺CD8⁺ cells are characterized by surface expression of a plurality of immune checkpoint molecules selected from the group consisting of CTLA-4, PD-1, TIGIT, Lag-3 and Tim-3. In another embodiment, said CD3⁺CD8⁺ cells are characterized by lack of substantial surface expression of CTLA-4. In another embodiment, about 50-51% of the CD3⁺CD8⁺ cells are characterized by surface expression of Lag-3. In another embodiment, about 50-54% of the CD3⁺CD8⁺ cells are characterized by surface expression of Tim-3. In another embodiment, about 15% of the CD3⁺CD8⁺ cells are characterized by surface expression of PD-1. In another embodiment, about 20-22% of the CD3⁺CD8⁺ cells are characterized by surface expression of TIGIT. Thus, ACT cell compositions according to embodiments of the invention comprise a CD3⁺CD8⁺ cell population defined as CTLA-4⁻, PD-1⁻, TIGIT⁻, Lag-3⁺ and Tim-3⁺. In another embodiment, ACT cell compositions according to embodiments of the invention comprise a CD3⁺CD8⁺ cell population defined as CTLA-4⁻, PD-1⁻, and TIGIT⁻. Each possibility represents a separate embodiment of the invention.

In some embodiments, the cell composition exhibits MHC-dependent (or MHC restricted) cytotoxic activity against hematopoietic tumor cells. In other embodiments, the cell composition exhibits MHC-independent (or non-MHC restricted) cytotoxic activity against hematopoietic tumor cells and solid tumor cells. In another embodiment, the cytotoxic activity is mediated by granzyme B and/or perforin. In another embodiment, said cytotoxic activity is not substantially mediated by FAS-FasL interactions. In another embodiment, said cell composition possesses both MHC-dependent and MHC-independent cytotoxic activities.

In another embodiment the composition is capable of specifically eradicating hematopoietic tumor cells in a significant manner when incubated with the tumor cells in vitro at a ratio of 0.25:1 for 24 hours. In another embodiment, the composition is capable of specifically eradicating hematopoietic tumor cells such that 80-90% of the tumor cells are eradicated upon incubation in vitro for 24 hours at a ratio of composition cells to tumor cells of 1:1. In another embodiment, the tumor cells express at least one NKG2D ligand. In another embodiment, the tumor cells express a plurality of NKG2D ligands selected from the group consisting of: MICA, MICB, HLAE, ULBP1, ULBP256, and ULBP3.

In another embodiment, the composition is capable of specifically eradicating non-histocompatible tumor cells in vivo without substantially eliciting GVHD. In another embodiment the composition is capable of inhibiting or preventing tumor development in a non-histocompatible subject in vivo without substantially eliciting GVHD.

In another embodiment, said composition retains at least 70% and typically at least 80% anti-tumor cytotoxic activity following cryopreservation and a subsequent thawing protocol.

In another embodiment, the composition is prepared by a process as disclosed herein. According to some embodiments, the cell composition is prepared by a process comprising: expanding PBMC in the constant presence of effective amounts of IL-2 and a CD3 activator for at least 9 days, wherein effective amounts of the IL-2 and CD3 activator are supplemented every 2-4 days. In another embodiment, incubation of cell composition is performed in the absence of other cytokines or antibodies.

In another embodiment, the cell composition is genetically modified. In another embodiment, the cell composition is genetically modified to expresses a CAR. In another embodiment the CAR is directed to an antigen selected from the group consisting of: BCMA, CD47, PDL-1, mesothelin, EpCAM, CD34, CD44, PSCA, MUC16, CD276, CD123, CD19, CD20 and EFFRvIII. In a particular embodiment the cells of the composition express a CD19-specific CAR or a CD123-specific CAR. In another embodiment, the cell composition is genetically modified to express at least one cytokine, chemokine, or a receptor thereof. In another embodiment, the cell composition is genetically modified to express a modified chemokine receptor. In another embodiment, the modified chemokine receptor is a CXCR4 receptor comprising a mutation or truncation at the C-terminal tail domain. In another embodiment the cell composition expresses exogenously IL-2, IL-15 and/or IL-21. In a particular embodiment, the cell composition has been modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21.

In another embodiment, the method further comprises cryopreservation of the cell composition resulting from step c. In another embodiment, said process further comprises subjecting the cryopreserved cell composition to a thawing protocol (e.g. prior to administration to a subject in need thereof). In another embodiment, the method further comprises, following step c., cryopreservation and a subsequent thawing protocol, wherein said cell composition retains at least 70% and typically at least 80% of the anti-tumor cytotoxic activity characteristic of said cell composition prior to cryopreservation.

In another aspect, there is provided a cell composition comprising leukocytes that have been genetically modified to express: (i) a CD19-specific CAR or a CD123-specific CAR; (ii) a modified CXCR4 receptor comprising a mutation or truncation at the C-terminal tail domain; and (iii) at least one cytokine selected from the group consisting of IL-2, IL-15 and IL-21. In another embodiment, the cell composition is characterized in that 70-80% of the cells are CD3⁺CD8⁺ cells expressing NKG2D and granzyme B, at least 70% are CD56⁻ cells, and up to 5% are CD3⁻ cells. In another embodiment, the cell composition is prepared by a process comprising expanding PBMC in the constant presence of effective amounts of IL-2 and a CD3 activator for at least 9 days, wherein effective amounts of the IL-2 and CD3 activator are supplemented every 2-4 days.

In some embodiment, a cell composition of the invention as disclosed herein is for use in treating a tumor in a subject in need thereof. In one embodiment, the tumor is a hematopoietic tumor. In another embodiment, the tumor is a solid tumor. In some embodiments, said tumor is selected from the group consisting of leukemia, multiple myeloma, a prostate tumor or mesothelioma. In another embodiment said tumor is characterized by expression of at least one NKG2D ligand. In another embodiment said tumor cells express a plurality of NKG2D ligands selected from the group consisting of: MICA, MICB, HLAE, ULBP1, ULBP256, and ULBP3. In another embodiment said tumor cells express MICA, MICB, HLAE, ULBP1, ULBP256, and ULBP3.

In another embodiment, said tumor is characterized by expression of at least one chemokine selected from the group consisting of CXCR6 ligands, CXCR4 ligands, CXCR3 ligands and CXCR2 ligands. In another embodiment, said tumor is characterized by expression of at least one chemokine selected from the group consisting of CXCR6 ligands, CXCR4 ligands, and CXCR3 ligands. In another embodiment, said tumor is characterized by expression of CXCR6 ligands. In a particular embodiment, said tumor is characterized by expression of the CXCR6 ligand CXCL16.

In another embodiment, said tumor is characterized by expression of at least one inhibitory immune checkpoint molecule and/or ligand thereof. In some embodiments, said tumor may express at least one ligand of CTLA-4, PD-1, TIGIT, Lag-3, Tim-3 and combinations thereof, wherein each possibility represents a separate embodiment of the invention. In another embodiment, said tumor is characterized by expression of at least one ligand of an inhibitory immune checkpoint molecule selected from the group consisting of CTLA-4, PD-1, and TIGIT. In another embodiment, said tumor is characterized by expression of ligands of a plurality of inhibitory immune checkpoint molecule selected from the group consisting of CTLA-4, PD-1, and TIGIT. In another embodiment, said tumor is characterized by expression of at least one CTLA-4 ligand (e.g. CD80 and/or CD86).

In another embodiment said tumor is resistant to treatment by at least one checkpoint molecule inhibitor. In some embodiments, the tumor may be resistant to treatment by inhibitors of at least one checkpoint molecule selected from the group consisting of CTLA-4, PD-1, TIGIT, Lag-3, Tim-3 and combinations thereof, wherein each possibility represents a separate embodiment of the invention. In another embodiment, said tumor is resistant to treatment by inhibitors of at least one checkpoint molecule selected from the group consisting of CTLA-4, PD-1, and TIGIT. In another embodiment, said tumor is resistant to treatment by inhibitors of CTLA-4, PD-1, and TIGIT.

For example, cell compositions of the invention were exemplified herein to lack substantial expression of CTLA-4, and accordingly may be particularly advantageous for the treatment of tumors resistant to therapy by CTLA-4 blocking antibodies such as ipilimumab and/or tremelimumab, even as a sole therapeutic agent. Thus, in another embodiment, the subject is not under treatment regimen with checkpoint molecule inhibitors.

In another example, cell compositions of the invention may be used in the treatment of tumors expressing Tim-3 and/or Lag-3, e.g. as combination therapy with Tim-3 inhibitors and/or Lag-3 inhibitors, respectively. Thus, in other embodiments, the subject is under treatment regimen with at least one with checkpoint molecule inhibitor, e.g. directed to PD-1, TIGIT, Lag-3, Tim-3 or combinations thereof, wherein each possibility represents a separate embodiment of the invention. In another embodiment, the use further comprises administration of at least one checkpoint molecule inhibitor e.g. directed to PD-1, TIGIT, Lag-3, Tim-3 or combinations thereof, wherein each possibility represents a separate embodiment of the invention. In another embodiment, the use further comprises administration of at least one checkpoint molecule inhibitor directed to Lag-3, Tim-3 or combinations thereof. In some embodiments, use of cell compositions in accordance with the invention may allow for reducing or minimizing the dosage of an existing anti-cancer therapy, e.g. an immune checkpoint inhibitor.

In another aspect there is provided a method of treating a tumor in a subject in need thereof, comprising contacting the tumor cells with a therapeutically effective amount of a cell composition of the invention as disclosed herein.

In one embodiment, the cell composition is autologous, histocompatible allogeneic, or non-histocompatible allogeneic to said subject. In a particular embodiment said cell composition is non-histocompatible allogeneic to said subject. In another embodiment the contacting is performed in vivo. In another embodiment the contacting is performed ex vivo. In another embodiment the tumor is a hematopoietic tumor. In another embodiment the tumor is a solid tumor. In another embodiment said tumor is characterized by expression of at least one NKG2D ligand. In another embodiment said tumor cells express a plurality of NKG2D ligands selected from the group consisting of: MICA, MICB, HLAE, ULBP1, ULBP256, and ULBP3. In another embodiment said tumor cells express MICA, MICB, HLAE, ULBP1, ULBP256, and ULBP3.

The following examples are presented in order to more fully illustrate some embodiments of the invention. They should, in no way be construed, however, as limiting the broad scope of the invention.

### EXAMPLES

### Example 1. Generation and Characterization of Super-Activated Killer (SAK) Cells

Peripheral blood mononuclear cells (PBMC) were isolated from human blood by Ficoll-Hypaque density centrifugation (Sigma). PBMC were resuspended in RPMI medium (Complete medium+MEM NEAA ×1 and 0.1 mM 2-mercaptoethanol) at a concentration of 1×10⁶ cells/ml and stimulated on day 0 with 1000 IU/ml recombinant human IL-2 (rhIL-2, R&D Systems) and 30 ng/ml anti human CD3 antibody (OKT-3, eBioscience). The culture was maintained by addition of fresh medium+rhIL-2 and anti-CD3 at days 4, 6 and 8. The cells were expanded for at least 11 days, and harvested at different time points for further analysis.

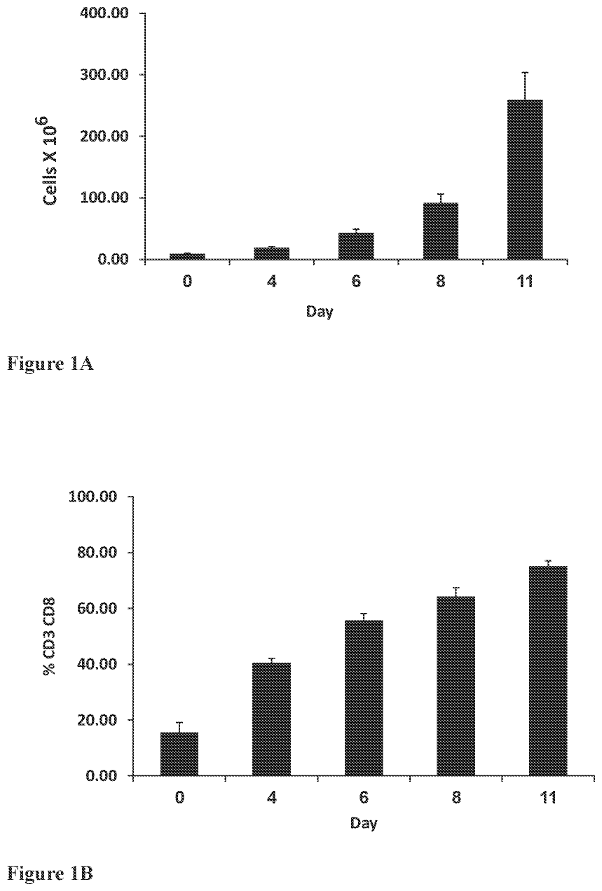

To evaluate the expansion rate, the harvested cells were stained by Trypan blue, and total live cells were counted at days 0, 4, 6, 8 and 11. The results are shown in FIG. 1A, in which mean±standard deviation from nine separate experiments are presented. As can be seen in FIG. 1A, cells were constantly expanded, reaching a 30.55-fold expansion rate after 11 days of culturing.

Next, immunophenotypic analysis by flow cytometry was performed as follows. Cells were harvested at different time points, washed, stained with various monoclonal antibodies at 4° C. for 30 min, and analyzed by flow cytometry. The following antibodies were used: FITC anti-CD4, APC anti-CD8, Percp anti-CD3, and PE anti-CD56 (all from Biolegend). The results are shown in FIGS. 1B-1D, in each of which means±standard deviations from nine separate experiments are shown.

As can be seen in FIGS. 1B-1D, the proportion (%) of CD3⁺CD8⁺ and CD3⁺CD56⁺ cells of the total expanded cell population increased gradually over time, while that of CD3⁺CD4⁺ cells decreased over time. Table 1 summarizes the mean values and proportions of the different sub-populations at day 11 (mean±SD of nine independent experiments). Table 2 further characterizes the sub-populations at day 11 (% of each sub population from the total composition, mean±SD of five independent experiments). CD8⁺CD56⁺/CD8⁺ represent the percentile of CD8⁺CD56⁺ out of the total CD8⁺ population.

TABLE 1
Characterization of cell composition at day 11

| | Fold | | | |
|---|---|---|---|---|
| No. of | change | | | |
| expanded | (day 11/ | % | % | % |
| cells | day 0) | CD3⁺CD8⁺ | CD3⁺CD4⁺ | CD3⁺CD56⁺ |
| 258 × 10⁶ ± | 30.55 ± | 75.04 ± | 9.92 ± | 24.44 ± |
| 45.6 | 4.01 | 2.02 | 0.54 | 3.08 |

TABLE 2
Characterization of sub-populations at day 11

| CD3⁺ | CD3⁺CD56⁺ | CD3⁺CD8⁺ | CD3⁺CD4⁺ | CD3−CD56⁺ | CD56⁺ | CD8⁺CD56⁺/CD8⁺ |
|---|---|---|---|---|---|---|
| 96.52 ± | 23.37 ± | 77.07 ± | 15.37 ± | 1.97 ± | 22.65 ± | 21.87 ± |
| 0.78 | 1.75 | 4.28 | 1.93 | 0.61 | 1.89 | 2.7 |

From a comprehensive analysis of samples obtained from twenty-five individual donors, the compositions were characterized by an average of 74.6±1.6% CD3⁺CD8⁺ cells and of 12±1% CD3⁺CD4⁺ cells. further, the ratio of CD3⁺CD8⁺ cells to CD3⁺CD4⁺ cells was at least 6:1 and typically about 7-8:1. The resulting cells, herein denoted super-activated killer (SAK) cells, were subjected to further analysis, as detailed in the subsequent Examples below.

In comparison, another known protocol for preparing PBMC-derived effector cells for ACT, namely cytokine-induced killer cells (CIK), was also tested. For the preparation of CIK, PBMC were resuspended in RPMI medium (Complete medium+MEM NEAA ×1 and 0.1 mM 2-mercaptoethanol) at a concentration of 1×10⁶ cells/ml and stimulated on day 0 with 1000 IU/ml recombinant human IFN-γ (Peprotec). On day 1, 50 ng/ml anti human CD3 antibody (OKT-3, eBioscience) and 500 IU/ml rhIL-2 (R and D system) were added. The culture was maintained by addition of fresh medium+500 IU/ml rhIL-2 at days 4, 6 and 8. The cells were expanded for at least 11 days. At day 11 CIK cells were harvested for further assay. SAK cells were also concomitantly expanded as detailed above. Table 3 provides the characterization of the CIK populations at day 11.

TABLE 3
Characterization of CIK cells at day 11

| Parameter | CIK |
|---|---|
| Fold change (day 11/day 0) | 33 |
| % CD3⁺CD8⁺ | 60.32 |
| % CD3⁺CD4⁺ | 21.95 |
| % CD8⁺CD56⁺/CD8⁺ | 13.49 |

As can be seen from Tables 1-3, SAK cells are phenotypically distinct from CIK cells at day 11 of expansion, wherein the composition of SAK cells includes a significantly higher proportion of CD8⁺CD56⁺ cells out of the total CD8⁺ cells than in the CIK cell composition, and significantly lower proportions of CD3⁺CD4⁺ cells than in the CIK cell composition.

Further, CIK cells intended for ACT are further distinguished from the SAK cells described herein by the relative proportion of additional subpopulations, including, but not limited to the incidence of CD3⁻ cells and CD56⁺ cells, which are higher in CIK cells. CIK cell compositions are typically administered to cancer patients following 21 days of expansion, by a process essentially corresponding to the CIK protocol described above. Such compositions are typically characterized by an average content of at least 30% CD56⁺ cells, and more typically up to about 50% to 80% CD56⁺ cells, and by an average content of at least 5% and more typically up to about 10% to 70% CD3⁻ cells. For instance, various hitherto reported exemplary CIK compositions were characterized at day 21 as comprising: 40-80% CD3⁺CD56⁺ cells, 20-60% CD3⁺CD56⁻ cells, and 1-10% CD3⁻CD56⁺ cells; 35% CD3⁺CD56⁺ cells, 65% CD3⁺CD56⁻ cells, and 2.23% CD3⁻CD56⁺ cells (averaged values, 90% total CD3⁺ cells); or 31.6% CD3⁺CD56⁺ cells, 41.5% CD3⁺CD56⁻ cells, and 11.7% CD3⁻CD56⁺ cells (median values).

### Example 2. Anti-Tumor Cytotoxic Activity of SAK Cells

To assess the impact of the effector cells (SAK) against various tumor cells (also referred to herein as target cells), cytotoxicity assay was performed using carboxyfluorescein diacetate succinimidyl ester (CFSE)-based assay. SAK cells were harvested at day 11 of culture. Target cells were labeled with CFSE (eBioscience) according to the manufacturer's instruction and plated at a concentration of 2×10⁴ cells/well in 96 well plate. The effector cells were added to the target cells at effector-to-target (E:T) ratios of 0.25:1, 0.5:1, 1:1, 2:1, 5:1, 10:1 and 30:1. After 24 h, the CFSE⁺PI⁻ labeled target cells were analyzed by flow cytometry. The percentile of live cells were calculated as: (the number of CFSE⁺PI⁻ cells with effector cells/the number of CFSE⁺PI⁻ cells without effector cells)×100.

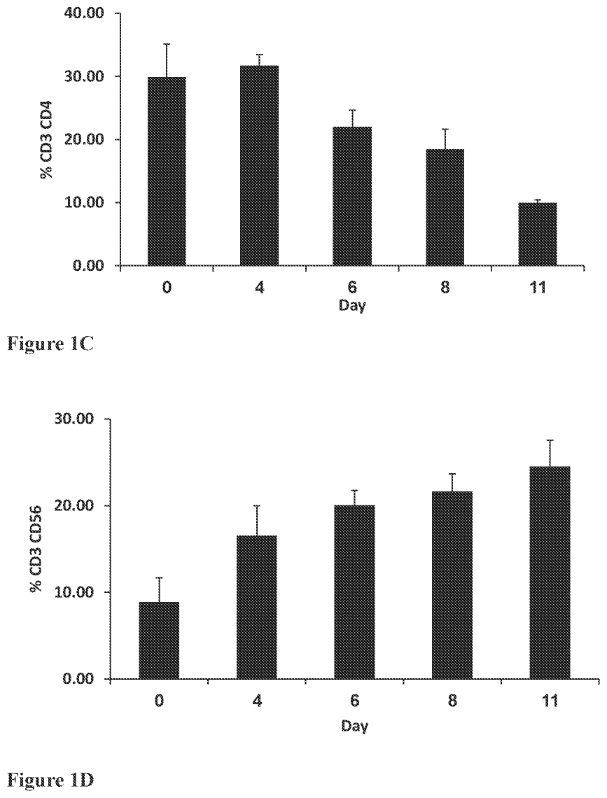

The results obtained with THP-1 leukemia cells and RPMI 8266 multiple myeloma cells are shown in FIGS. 2A-2B, respectively, in which means±standard deviation from triplicate at each ratio are presented. As can be seen in FIGS. 2A-2B, SAK cells demonstrated a potent cytotoxic activity against both types of hematopoietic tumor cell lines, THP-1 and RPMI 8266, even at E:T ratios as low as 0.25:1. When used at an E:T ratio of 2:1, SAK cells showed remarkable killing efficiency of about 90%, indicating the potency of SAK cells as anti-tumor cell therapy.

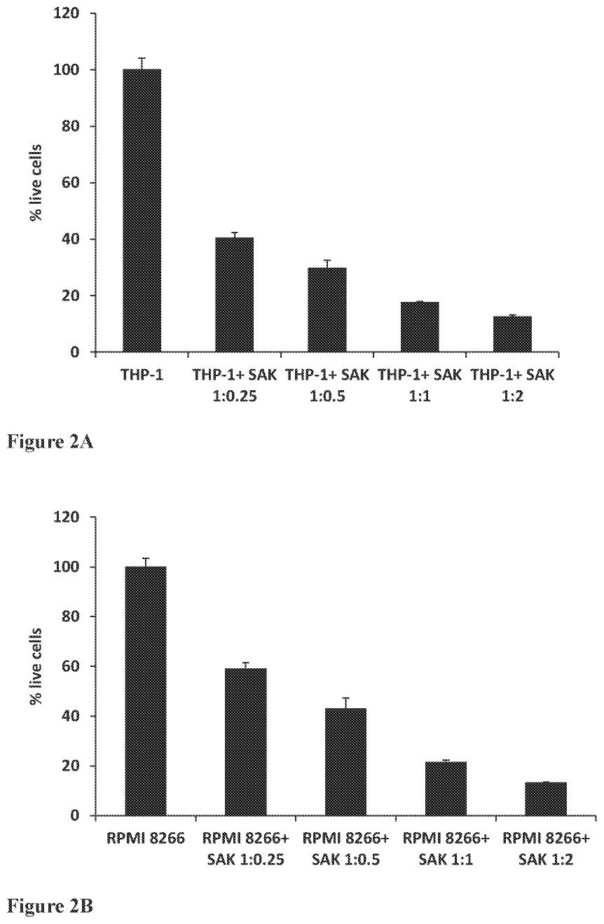

The experiments were repeated for a variety of solid tumor cell lines, including PC3 prostate cancer cells and H28 mesothelioma cancer cells, and compared to hematopoietic MV4-11 leukemia cells. The results are shown in FIGS. 3A-3C in which means±standard deviation from triplicate at each ratio are presented. As can be seen in FIGS. 3A-3C, SAK cells showed marked potency against the hematopoietic MV4-11 cells and against both solid tumor cell lines. Increased E:T ratios resulted in enhanced efficacy of the SAK cell against all cancer cell lines, and especially against MV4-11 leukemia cells. For example, E:T ratio of 2:1 was required for 90% efficiency against MV4-11 cells, whereas an E:T ratio of 30:1 was required for 90% efficiency against PC3 prostate cancer cells and H28 mesothelioma cancer cells.

Next, the SAK cell composition was separated into CD3⁺CD56⁺ cells and CD3⁺CD56⁻ cells using flow cytometry, and the cytotoxic activity of each population compared to that of the original SAK cell composition. To this end, each population was incubated with MV4-11 cells as detailed above, at various E:T ratios (0.25:1, 0.5:1, 1:1 and 2:1). Notably, no significant differences were found between the cytotoxic capacity of the tested populations and that of the complete SAK composition, in any on the E:T ratios tested. Further, similar results were obtained using SAK cell compositions harvested at days 10 and 12 of expansion.

Thus, SAK cells exhibited potent anti-tumor cytotoxic activity against a variety of tumor cells in an MHC II-independent manner. Further, the cytotoxic activity appears to be CD56⁻ independent.

### Example 3. SAK Cells Exhibit Significantly Improved Anti-Tumor Cytotoxic Activity Compared to Other PBMC-Derived Effector Cells

Following the discovery that SAK cells exert an excellent anti-tumor activity, cytotoxic activity of SAK cells was compared to two other PBMC-derived effector cells, namely cytokine-induced killer cells (CIK) and activated lymphocyte compositions (IL-2-expanded T cells).

For the preparation of CIK, PBMC were resuspended in RPMI medium (Complete medium+MEM NEAA ×1 and 0.1 mM 2-mercaptoethanol) at a concentration of 1×10⁶ cells/ml and stimulated on day 0 with 1000 IU/ml recombinant human IFN-γ (Peprotec). 50 ng/ml anti human CD3 antibody (OKT-3, eBioscience) and 500 IU/ml rhIL-2 (R and D system) were added on day 1. The culture was maintained by addition of fresh medium+500 IU/ml rhIL-2 at days 4, 6 and 8. The cells were expanded for at least 11 days. At day 11 CIK cells were harvested for further assays. IL-2-expanded T cells cultures were obtained after incubation of PBMC with 50 ng/ml anti human CD3 and 300 IU/ml rhIL-2 at day 0 and further addition of fresh medium with 300 IU/ml rhIL-2.

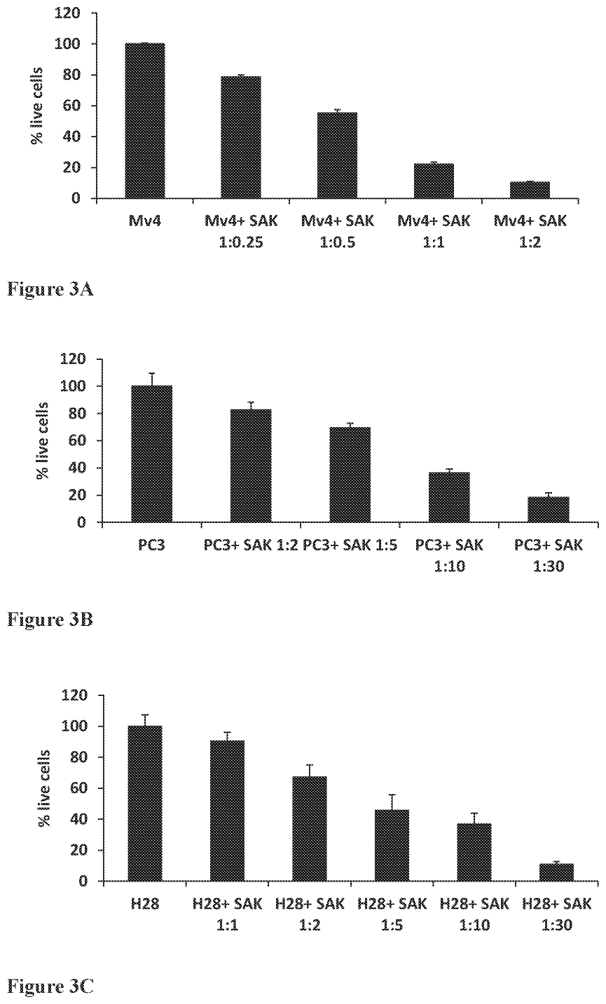

CFSE-based cytotoxicity assays were performed essentially as described in Example 2, using SAK, CIK or IL-2-expanded T cells as effector cells, and MV4-11 leukemia cells as the target cells, when untreated MV4-11 cells were set as control. The results are shown in FIGS. 4A-4B, in which means±standard deviation from triplicate at each ratio are presented.

Unexpectedly, as can be seen in FIGS. 4A-4B, SAK cells were found to be significantly more effective in eradicating the tumor cells than the control effector cells. When compared to CIK cells (FIG. 4A), lower E:T ratios were required for SAK cells to exhibit a significant cytotoxic activity, wherein SAK cells exhibited a superior cytotoxic activity at each tested E:T ratios. For example, at an E:T ratio of 5:1, SAK cells exhibited 90% efficiency while CIK cells at the same ratio showed efficiency of only 40% (FIG. 4A). SAK cells were found to be even more effective compared to IL-2-expanded T cells. For example, even at an E:T ratio of 0.25:1, SAK cells displayed 60% efficiency while the IL-2-expanded T cells at the same ratio were completely ineffective; even at an E:T ratio of 2:1, SAK cells exhibited over 90% killing activity while IL-2-expanded T cells exhibited merely 25% activity (FIG. 4B).

Thus, SAK cells were unexpectedly found to exert an extraordinarily efficient anti-tumor cytotoxic activity compared to hitherto known PBMC-derived effector cells.

### Example 4. Immunophenotypic Characterization of SAK Cells and Tumor Cells

Expression of NKG2D on CD8⁺ SAK cells was evaluated as follows. SAK cells prepared as described in Example 1 were harvested at day 11 of expansion, washed, stained with FITC-labeled anti-NKG2D and APC-labeled anti-CD8 monoclonal antibodies (Biolegend) at 4° C. for 30 min, and analyzed by flow cytometry. The staining by the antibodies on CD8⁺ cells was detected and compared with isotype antibody control staining.

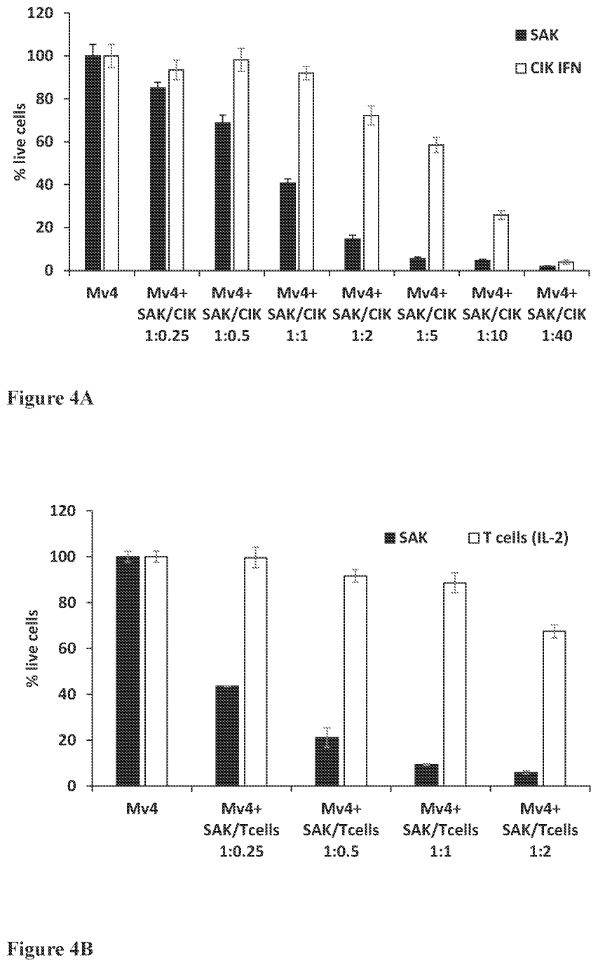

The results are shown in FIG. 5. As can be seen in FIG. 5, CD8⁺ SAK cells highly express NKG2D. Remarkably, 100% of the CD8⁺ cells were found to express NKG2D, with an 8.4-fold enhancement in mean fluorescent intensity over the isotype control.

Since SAK cells showed expression of the NKG2D receptor, the expression of various NKG2D ligands was tested on different types of tumor cells using flow cytometry. The results are summarized in Table 4 below.

TABLE 4
Expression of various NKG2D ligands by leukemia
MV4-11 cells and H28 mesothelioma cancer cells

| Ligand | MV411 | H28 |
|---|---|---|
| MICA/B | + | − |
| HLAE | + | − |
| ULBP1 | + | − |
| ULBP256 | + | − |
| ULBP3 | + | − |

As can be seen in Table 4, MV411 leukemia cells highly express a variety of NKG2D ligands including ULBP3, HLAE, ULBP1, ULBP256, and MICA and/or MICB on their surface. In contrast, H28 mesothelioma cells express these markers at significantly lower, non-significant levels (e.g. ULBP256), or even non-detectable levels (e.g. MICA and MICB).

Thus, SAK cells were found to be characterized by high NKG2D expression, and to be particularly effective against target tumor cells characterized by high expression of NKG2D ligands such as MV4-11 leukemia cells.

### Example 5. Cytotoxic Activity of SAK is Mediated Mainly by Granzyme B and Perforin Secretion and not by FasL-Fas Interaction

In order to examine the killing mechanism of the SAK cells, expression levels of FASL, granzyme B and perforin on the CD8+ SAK cells were examined essentially as described in Example 4. Briefly, SAK cells were harvested at day 11 of expansion, washed, stained with PE anti-FasL APC anti perforin or PE anti-granzyme monoclonal antibodies (all from Biolegend) at 4° C. for 30 min, and analyzed by flow cytometry. The staining by the antibodies on CD8⁺ cells was detected and compared with isotype antibody control staining.

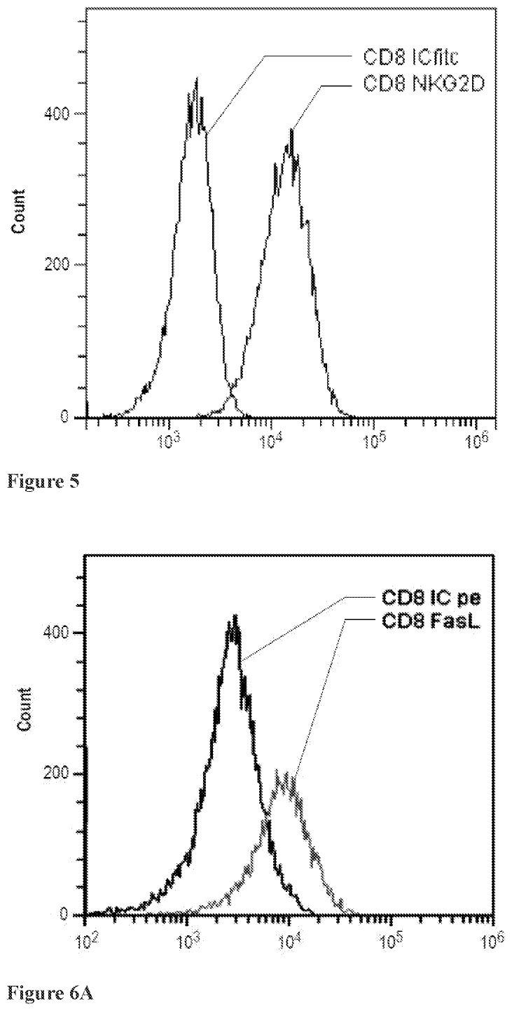

The results shown in FIGS. 6A-6C demonstrate high expression of FasL, Granzyme B and perforin by the CD8⁺ cytotoxic cell population. Table 5 below summarizes the expression levels, represented as mean fluorescent intensity (MFI), on CD8⁺ cells stained with marker-specific antibodies compared to that of isotype control antibody. As can be seen in Table 5, 40% of the CD8⁺ cells are FasL positive, 50% are perforin positive and 100% express Granzyme B.

TABLE 5
Expression FasL, Granzyme B and Perforin on CD8⁺ SAK cells

| | FasL | Granzyme B | Perforin |
|---|---|---|---|
| MFI isotype control | 3102 | 2056 | 6929 |
| MFI antibody | 9476 | 112952 | 16059 |
| Expressing cells (%) | 40 | 100 | 50 |

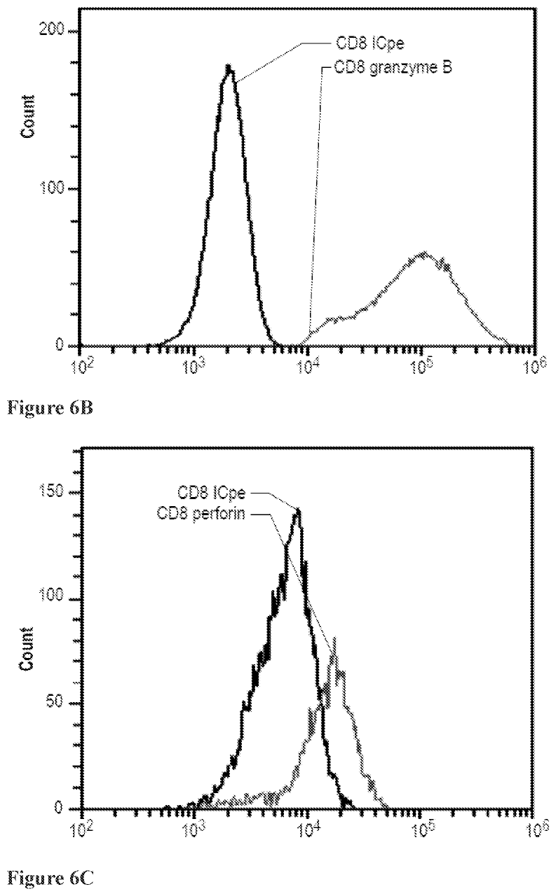

Next, target-induced perforin secretion was examined, as follow. An amount of 5×10³ SAK cells (at day 11) was added to MV4-11 cells at a ratio of 0.25:1 (E:T) for 24 h. After 24 h, the concentrations of perforin in the culture medium supernatant were examined using an enzyme-linked immunosorbent assay (ELISA) kit (Abcam). As can be seen in FIG. 7, perforin secretion levels increased following the incubation with the target MV4-11 tumor cells.

In order to further characterize the killing mechanism, a CFSE-based cytotoxicity assay was performed essentially as described in Example 2, in the presence of various inhibitors. To this end, leukemia MV4-11 cells were cultured with SAK cells with and without the tested inhibitor, and untreated MV4-11 cells were set as control. The following inhibitors were used: 3,4-Dichloroisocoumarin (DCI—granzyme B inhibitor, Sigma), Concanamycin A (CMA—perforin inhibitor, Sigma) and anti-FasL blocking antibody (Biolegend). 50 μM DCI, 1000 mM CMA or 5 μg/ml anti FasL were added to SAK cells 2 h before incubation with target cells. The cytotoxic activity of SAK against the leukemic MV4-11 target cells was expressed as a percentage of live cells as calculated by (the number of CFSE⁺PI⁻ cells in the presence of effector cells/the number of CFSE⁺PI⁻ cells without effector cells)×100.

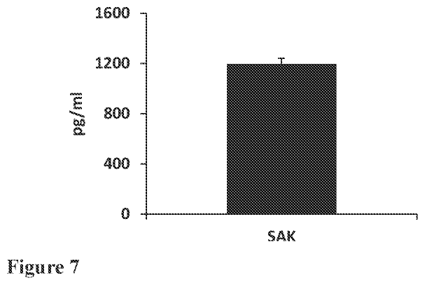

The results are shown in FIGS. 8A-8C, in which means±standard deviation from triplicate at each ratio are presented. As can be seen in the figure, granzyme B inhibitor and perforin inhibitor significantly reduced the cytotoxic activity of SAK cells (FIGS. 8A and 8C, respectively) while incubation with the anti-FasL antibody had little or no effect on reducing the percentage of living cells (FIG. 8B).

Thus, without wishing to be bound by a specific theory or mechanism of action, the SAK cells appear to exhibit an anti-tumor cytotoxic activity which is mainly dependent on Granzyme B and perforin secretion rather than on FasL-Fas interaction.

### Example 6. Characterization of SAK Migration Ability

In order to further characterize the SAK cell populations, analysis of chemokine receptor expression was performed by flow cytometry, as follows. Cells were harvested at different time points, washed, stained with APC anti-CXCR4 (12G5 clone), APC anti-CXCR3, PE anti-CXCR6 or FITC anti-CXCR2 monoclonal antibodies, as well as with anti-CD8 antibodies labeled with FITC or APC, respectively (all from Biolegend) at 4° C. for 30 min, and analyzed by flow cytometry.

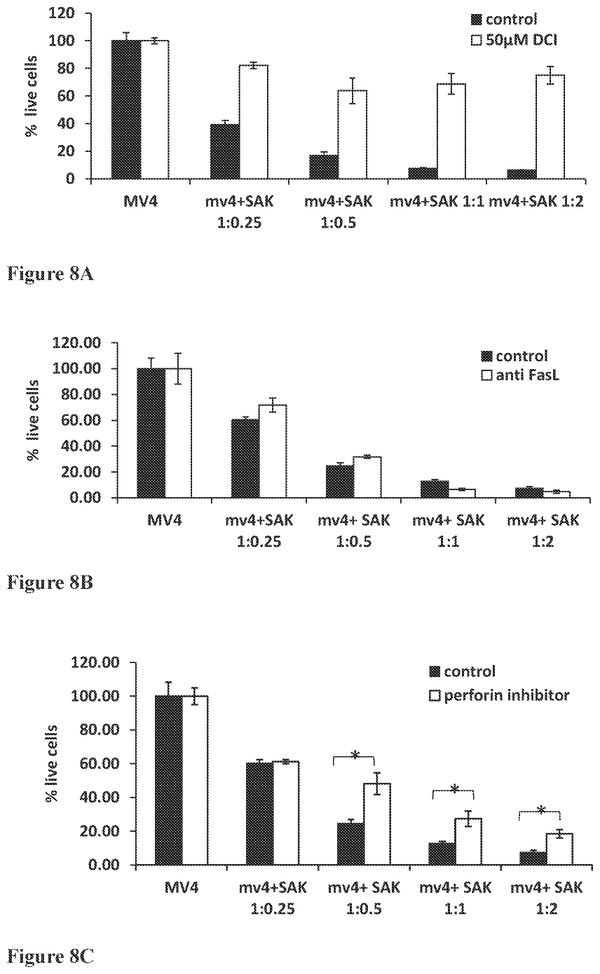

The results shown in FIG. 9A show the expression of the chemokine receptors CXCR2, CXCR3 (top panel, left and right respectively), CXCR4 and CXCR6 (bottom panel, left and right respectively) on SAK cells at day 11 of expansion. In particular, substantially all CD3⁺CD8⁺ cells were found to be characterized by surface expression of CXCR4, and the remaining chemokine receptors were found, on average, on about 90% (CXCR2 and CXCR3), or about 30% (CXCR6) of the CD3⁺CD8⁺ cells.

In order to test the functionality of the chemokine receptors on the SAK cells, migration assays were performed. To this end, migration of SAK cells in response to the chemokines CXCL12, IP-10 or rhIL-8 (PeproTech EC) was evaluated using 5-μm pore size Transwells (Costar). SAK cells were resuspended in RPMI medium containing 1% Fetal Calf Serum (FCS). Cells (2×10⁵ cells/well) were added to the upper chambers in a total volume of 100 μL, and 600 L RPMI supplemented with 100 ng/mL CXCL12 or IL-8, or 500 ng/ml IP-10 was added to the lower chambers. The quantity of cells migrating within four hours to the lower compartment was determined by FACS and expressed as a percentage of the input cells in the upper chamber.

The results shown in FIG. 9B demonstrate that SAK cells migrate toward both the chemokines IL-8, SDF-1 and IP-10. In particular, 3% of the cells migrated towards IL-8 at the tested concentration, whereas about 40% of the cells migrated towards SDF-1 and about 30% migrate toward IP-10. Thus, CXCR4 and CXCR3-dependent migration appeared to be more pronounced than CXCR2-dependent migration in the SAK populations. The expression of CXCR3 and CXCR6 on SAK cells indicates that SAK cells have also potential to migrate towards CXCR3 ligands CXCL9 and CXCL11, and CXCR6 ligand CXCL16.

In summary, the results demonstrate the ability of SAK cells to be recruited to tumor tissue, in particular to tumors expressing CXCR4, CXCR6 and CXCR6 ligands. Of additional interest is the observation that certain chemokine receptors such as CXCR6 were suggested to be particularly important for the survival and expansion of cytotoxic T cells at the tumor microenvironment, thereby reducing T cell exhaustion. Accordingly, and without wishing to be bound by a specific theory or mechanism of action, expression of CXCR6 on SAK cells may represent an activated phenotype, and an enhanced potential for survival and expansion at the tumor microenvironment.

### Example 7. Adoptive Transfer In Vivo

To generate the xenograft model, NOD SCID gamma (NSG) mice (immunodeficient mice lacking lack mature T cells, B cells, and NK cells) were irradiated with 300 cGy. 24 hrs later, RAJI cells (human B lymphoblastoid line) were intravenously (iv) injected (1×10⁵ cells/mouse) via the dorsal tail vein at a final volume of 200 μl.

Next, mice were injected intravenously (iv) with 20×10⁶ SAK cells and intraperitoneally (ip) with 10 μg IL-2 at days 5, 6 and 7. Control mice were injected with PBS iv and 10 μg IL-2 ip. Mice were sacrificed at day 12 and bone marrow (BM) and spleen were taken for analysis. Cells were isolated from those organs, stained with anti-human CD20 antibody (specifically recognizing the engrafted tumor cells), and the percentage of engrafted cells in the BM and spleen were evaluated by FACS. The results are presented in Table 6 below, in which % inhibition was calculated as 100−(CD20 in SAK mice/CD20 in control mice*100).

TABLE 6
% Raji cells (CD20⁺) in BM and spleen

| Group | BM | Spleen |
|---|---|---|
| Control-1 | 0.86 | 2.9 |
| Control-2 | 3.75 | 1.43 |
| Control-3 | 2.03 | 7.1 |
| Control-4 | 3.08 | |
| Control-5 | 1.94 | 3.7 |
| SAK-1 | 3.79 | 2.37 |
| SAK-2 | 1.05 | 0.85 |
| SAK-3 | 1.61 | 0.28 |
| SAK-4 | 2.17 | 0.37 |
| SAK-5 | 1.68 | 0.38 |
| Average Control | 2.332 ± 0.499 | 3.78 ± 1.20 |
| Average SAK | 2.06 ± 0.467 | 0.85 ± 0.39 |
| T-test | 0.35 | 0.04 |
| % inhibition | 11.66 | 77.51 |

As can be seen in Table 6, adoptive transfer of SAK cell significantly inhibited the engraftment of Raji cells in the bone marrow and spleen, with a particularly remarkable efficacy providing about 78% inhibition of tumor development in the spleen.

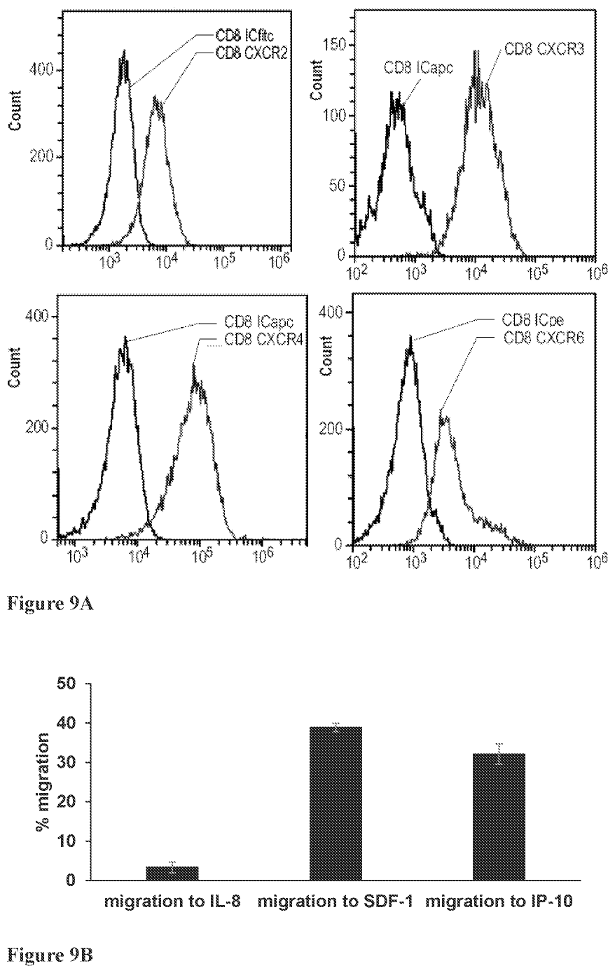

The experiment was repeated as detailed above, but with the BM and spleen cells, as well as blood taken from the sacrificed NSG mice stained with anti-human CD19 antibody. The results are presented in Table 7 below, in which % inhibition was calculated as 100−(CD19 in SAK mice/CD19 in control mice*100), and visualized in FIG. 10A-10C, which show the percentage of CD19⁺ Raji cells in control NSG mice compared to NSG mice after transfer of SAK cells in spleen (FIG. 10A), BM (FIG. 10B) and blood (FIG. 10C).

TABLE 7
% Raji cells (CD19⁺) in BM, spleen and blood

| Group | BM | Spleen | Blood |
|---|---|---|---|
| Control-1 | 1.65 | 13.67 | 0.29 |
| Control-2 | 2.7 | 6.94 | 0.14 |
| Control-3 | 2.13 | 14.3 | 2.28 |
| Control-4 | 1.49 | 4.67 | 2.56 |
| Control-5 | 1.65 | 12.67 | 0.31 |
| SAK-1 | 0.6 | 0.78 | 0.08 |
| SAK-2 | 0.64 | 0.01 | 0.22 |
| SAK-3 | 1.06 | 0.08 | 0.08 |
| SAK-4 | 0.71 | 0.24 | 0.23 |
| SAK-5 | 1.08 | 0.34 | 0.15 |
| Average Control | 1.92 | 10.45 | 1.12 |
| Average SAK | 0.82 | 0.29 | 0.15 |
| T-test | 0.0023 | 0.0031 | 0.073 |
| % inhibition | 57.48 | 97.22 | 86.38 |

As can be seen in Table 7, adoptive transfer of SAK cells significantly inhibited the engraftment of Raji cells in the bone marrow, blood and spleen, with a remarkable efficacy providing about 97% inhibition of tumor development in the spleen. These findings indicate that SAK cells alone are able to effectively inhibit tumor growth and engraftment.

### Example 8. Genetically Modified SAK

Improved, genetically modified, SAK cells were prepared as described below.

For the generation of nucleic acid constructs, the retroviral MSGV-1D3-28Z All ITAMs intact expression vector (#107226 from Addgene) was used, with the following modifications. The retroviral vector backbone, pMSGV1, is a derivative of the MSCV-based splice-GAG vector (pMSGV), which uses a murine stem cell virus (MSCV) long terminal repeat. The restriction enzyme NcoI and SalI were used in order to remove the “1D3-28Z All ITAMs intact” insert from the plasmid. Various cDNA inserts as detailed below were amplified by PCR and ligated to the plasmid with NcoI and SalI restriction sites. The resulting constructs A-D as detailed in Table 8 below were confirmed by DNA sequencing.

TABLE 8
Constructs for genetic modification

| | Insert (N′ to | |
|---|---|---|
| Construct | C′ order) | Expressing |
| A | Anti CD123-IRES-IL-2 | Human CD123-specific |
| | | CAR, human IL-2 |
| B | IL-2-IRES-GFP | Human IL-2, green |
| | | fluorescent protein GFP) |
| C | IL-15-2A-GFP | Human IL-15, GFP |
| D | CXCR4 whim | C′-truncated CXCR4 |

The nucleic acid sequence encoding the anti-human CD123 CAR (included in construct A) is as follows:

(SEQ ID NO: 1)
ATGGCCCTGCCTGTGACAGCCCTGCTGCTGCCTCTGGCTCTGCTGCTGCA
TGCCGCTAGACCCGATATTGTCCTCACTCAATCGCCGGACTCACTGGCGG
TGTCCCTCGGAGAGAGGGCGACGATCAATTGCCGGGCTTCCGAATCCGTC
GATAACTACGGAAACACCTTTATGCACTGGTACCAACAGAAGCCAGGACA
GCCACCAAAGCTGTTGATCTACCGCGCTTCAAACCTTGAGTCGGGTGTGC
CGGACCGCTTCAGCGGCAGCGGTTCCAGAACCGACTTTACCCTCACCATC
AGCTCGCTGCAGGCCGAAGATGTCGCCGTCTATTACTGCCAACAGAGCAA
CGAAGATCCGCCTACTTTCGGACAGGGGACTAAACTGGAAATCAAGGGCG
GAGGAGGCTCGGGTGGAGGAGGATCGGGAGGAGGCGGGTCCGGTGGTGGC
GGATCGCAAATCCAGCTGGTGCAGTCCGGCGCAGAAGTGAAGAAGCCGGG
AGCGTCCGTGAAAGTGAGCTGCAAGGCCTCAGGGTACATCTTCACCAATT
ACGGCATGAATTGGGTGCGGCAGGCACCCGGACAGCGCCTGGAGTGGATG
GGCTGGATCAACACTTACACCGGGGAAAGCACGTACTCGGCCGACTTCAA
AGGACGGGTGACCATTACCCTGGATACCTCGGCCTCAACCGCTTACATGG
AGCTCTCATCACTTAGATCCGAGGACACTGCCGTCTACTACTGTGCAAGG
AGCGGAGGCTACGACCCTATGGACTATTGGGGACAAGGCACTACTGTGAC
TGTGTCGTCCACCACGACGCCAGCGCCGCGACCACCAACACCGGCGCCCA
CCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTA
CATCTGGGCGCCCTTGGCCGGGACTTGTGGGGTCCTTCTCCTGTCACTGG
TTATCACCCTTTACTGCTAA.

The IRES nucleic acid sequence (included in constructs A and B) is as follows:

(SEQ ID NO: 2)
GAATTCCCGGGTCGACCTGCAGAAGCTTAAAACAGCTCTGGGGTTGTACC
CACCCCAGAGGCCCACGTGGCGGCTAGTACTCCGGTATTGCGGTACCCTT
GTACGCCTGTTTTATACTCCCTTCCCGTAACTTAGACGCACAAAACCAAG
TTCAATAGAAGGGGGTACAAACCAGTACCACCACGAACAAGCACTTCTGT
TTCCCCGGTGATGTCGTATAGACTGCTTGCGTGGTTGAAAGCGACGGATC
CGTTATCCGCTTATGTACTTCGAGAAGCCCAGTACCACCTCGGAATCTTC
GATGCGTTGCGCTCAGCACTCAACCCCAGAGTGTAGCTTAGGCTGATGAG
TCTGGACATCCCTCACCGGTGACGGTGGTCCAGGCTGCGTTGGCGGCCTA
CCTATGGCTAACGCCATGGGACGCTAGTTGTGAACAAGGTGTGAAGAGCC
TATTGAGCTACATAAGAATCCTCCGGCCCCTGAATGCGGCTAATCCCAAC
CTCGGAGCAGGTGGTCACAAACCAGTGATTGGCCTGTCGTAACGCGCAAG
TCCGTGGCGGAACCGACTACTTTGGGTGTCCGTGTTTCCTTTTATTTTAT
TGTGGCTGCTTATGGTGACAATCACAGATTGTTATCATAAAGCGAATTGG
ATTGCGGCCGC.

The nucleic acid sequence of human IL-2 (Accession No. BC066257; included in constructs A and B) is as follows:

(SEQ ID NO: 3)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTGACTTGA.

The nucleic acid sequence of human IL-15 (Accession No. BC100961.1; included in construct C) is as follows:

(SEQ ID NO: 4)
ATGAGAATTTCGAAACCACATTTGAGAAGTATTTCCATCCAGTGCTACTT
GTGTTTACTTCTAAACAGTCATTTTCTAACTGAAGCTGGCATTCATGTCT
TCATTTTGGGCTGTTTCAGTGCAGGGCTTCCTAAAACAGAAGCCAACTGG
GTGAATGTAATAAGTGATTTGAAAAAAATTGAAGATCTTATTCAATCTAT
GCATATTGATGCTACTTTATATACGGAAAGTGATGTTCACCCCAGTTGCA
AAGTAACAGCAATGAAGTGCTTTCTCTTGGAGTTACAAGTTATTTCACTT
GAGTCCGGAGATGCAAGTATTCATGATACAGTAGAAAATCTGATCATCCT
AGCAAACAACAGTTTGTCTTCTAATGGGAATGTAACAGAATCTGGATGCA
AAGAATGTGAGGAACTGGAGGAAAAAAATATTAAAGAATTTTTGCAGAGT
TTTGTACATATTGTCCAAATGTTCATCAACACTTCT.

The nucleic acid sequence of the 2A element (included in construct C) is as follows:

(SEQ ID NO: 5)
GAGGGCAGGGGAAGTCTACTAACATGCGGGGACGTGGAGGAAAATCCCGG
CCCA.

The nucleic acid sequence of GFP (included in constructs B and C) is as follows:

(SEQ ID NO: 6)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA.

The wild-type CXCR4 chemokine receptor has the following amino acid sequence:

(SEQ ID NO: 7)
MSIPLPLLQIYTSDNYTEEMGSGDYDSMKEPCFREENANFNKIFLPTIYS
IIFLTGIVGNGLVILVMGYQKKLRSMTDKYRLHLSVADLLFVITLPFWAV
DAVANWYFGNFLCKAVHVIYTVNLYSSVLILAFISLDRYLAIVHAINSQR
PRKLLAEKVVYVGVWIPALLLTIPDFIFANVSEADDRYICDRFYPNDLWV
VVFQFQHIMVGLILPGIVILSCYCIIISKLSHSKGHQKRKALKTIVILIL
AFFACWLPYYIGISIDSFILLEIIKQGCEFENTVHKWISITEALAFFHCC
LNPILYAFLGAKFKTSAQHALTSVSRGSSLKILSKGKRGGHSSVSTESES
SSFHSS.

A nucleic acid sequence encoding a modified, C′-truncated CXCR4 (WHIM R334x mutation, in which the 19 C-terminal amino acid of SEQ ID NO: 7 are removed, NM_001008540.2; included in construct D), has the following nucleic acid sequence:

(SEQ ID NO: 8)
ATGTCCATTCCTTTGCCTCTTTTGCAGATATACACTTCAGATAACTACAC
CGAGGAAATGGGCTCAGGGGACTATGACTCCATGAAGGAACCCTGTTTCC
GTGAAGAAAATGCTAATTTCAATAAAATCTTCCTGCCCACCATCTACTCC
ATCATCTTCTTAACTGGCATTGTGGGCAATGGATTGGTCATCCTGGTCAT
GGGTTACCAGAAGAAACTGAGAAGCATGACGGACAAGTACAGGCTGCACC
TGTCAGTGGCCGACCTCCTCTTTGTCATCACGCTTCCCTTCTGGGCAGTT
GATGCCGTGGCAAACTGGTACTTTGGGAACTTCCTATGCAAGGCAGTCCA
TGTCATCTACACAGTCAACCTCTACAGCAGTGTCCTCATCCTGGCCTTCA
TCAGTCTGGACCGCTACCTGGCCATCGTCCACGCCACCAACAGTCAGAGG
CCAAGGAAGCTGTTGGCTGAAAAGGTGGTCTATGTTGGCGTCTGGATCCC
TGCCCTCCTGCTGACTATTCCCGACTTCATCTTTGCCAACGTCAGTGAGG
CAGATGACAGATATATCTGTGACCGCTTCTACCCCAATGACTTGTGGGTG
GTTGTGTTCCAGTTTCAGCACATCATGGTTGGCCTTATCCTGCCTGGTAT
TGTCATCCTGTCCTGCTATTGCATTATCATCTCCAAGCTGTCACACTCCA
AGGGCCACCAGAAGCGCAAGGCCCTCAAGACCACAGTCATCCTCATCCTG
GCTTTCTTCGCCTGTTGGCTGCCTTACTACATTGGGATCAGCATCGACTC
CTTCATCCTCCTGGAAATCATCAAGCAAGGGTGTGAGTTTGAGAACACTG
TGCACAAGTGATTTCCATCACCGAGGCCCTAGCTTTCTTCCACTGTTGTC
TGAACCCCATCCTCTATGCTTTCCTTGGAGCCAAATTTAAAACCTCTGCC
CAGCACGCACTCACCTCTGTGAGCAGAGGGTCCAGCCTCAAGATCCTCTC
CAAAGGAAAGTAA.

For the preparation of retroviral particles for transduction, eco-phoenix cells were inoculated in 6-well plates at a density of 5×10⁵ cells and allowed to reach 60-70% confluence at the day of infection. The retrovirus plasmid MSGV (containing the tested construct) 2 μg/well, were transfected into Eco-phoenix cells using lipofectamine Transfection Reagent for 5 h at 37° C. in 5% CO₂, according to the manufacturer's protocol. Following 48 hr, supernatants containing retrovirus particles were harvested and filtered through a 0.45-μm filter (EMD Millipore, Billerica, MA, USA) to remove cell debris. The supernatants were used to transduce PG13 cells in order to generate a PG13-based packaging cell line, producing the modified MSGV virus.

PG13 transduction was mediated by RetroNectin® (Takara Shuzo). Non-tissue culture 24-well plates were coated with RetroNectin® (diluted 1:40 in PBS). Plates were coated overnight at 4° C., subsequently blocked with 2% BSA for 30 minutes at room temperature (RT), and washed once with PBS before use. Virus-containing supernatant from the transfected eco-phoenix cells was added to RetroNectin® coated plate and centrifuged for 2 h. In the next step, PG13 cells were added to the wells and incubated with the virus for 24 h. Three days later, positive PG13 cells (detected by FACS) were selected and expanded.

The resulting viral particles were used to transduce SAK cells prepared essentially as described in Example 1, during the expansion process. Specifically, freshly isolated PBMC were stimulated for 48 hr in the presence of 30 ng/mL of OKT3 and 1000 IU/ml IL-2. Following stimulation, cells were transduced with retroviral vectors (produced by the PG13 cells as described above) by transfer to culture dishes that had been precoated with retroviral vector as described above. To this end, non-tissue culture 24-well plates were coated with RetroNectin®, virus-containing supernatant (from PG13) was added to the RetroNectin®-coated plates and centrifuge for 2 h. In the next step, the stimulated cells were added to the wells and incubated with the viral particles for 24 h. After that, cells were further expanded with 30 ng anti CD3 and 1000 IU/ml IL-2 as described in Example 1. Positive (construct-containing) cells were detected and quantified by FACS using: APC anti-CXCR4 antibody, CD123-Fc and PE anti Fc antibody, or GFP analysis, and confirmed to express the corresponding exogenous genes.

Similarly, constructs A and D are cloned together with the 2A sequence into the plasmid backbone, to produce a nucleic acid construct capable of constitutively expressing the CD123-specific CAR, the modified CXCR4 receptor, and IL-2 (Anti CD123-IRES-IL-2-2A-CXCR4 whim). The combined construct is transduced to SAK cells as described above. Similarly, the construct CXCR4 whim-IRES-IL-2 is generated and transduced to freshly prepared SAK cells.

The resulting cells transduced with the various constructs are assayed for in vitro and in vivo anti-tumor activity essentially as described in Examples 2 and 7, respectively.

### Example 9. Cytotoxic Activity of Genetically Engineered SAK Cells Expressing CD19-Specific CAR

Genetically modified SAK cells expressing CD19 CAR (engineered to recognize CD19-expressing B cell malignancies) were prepared essentially as described in Example 8, with the following changes.

The vector was engineered to express a scFv targeting domain (specific to CD19), a CD8-derived hinge region, a CD8-derived transmembrane region, 4-1BB/CD137 costimulatory domain, and a CD3-zeta chain intracellular signaling domain. For the generation of nucleic acid constructs, the retroviral MSGV-1D3-28Z All ITAMs intact expression vector was used, with the CD19 CAR sequence. The restriction enzyme NcoI and SalI were used in order to remove the “1D3-28Z All ITAMs intact” insert from the plasmid. CD19 CAR cDNAs was amplified by PCR and ligated to the plasmid with NcoI and SalI restriction sites. The resulting constructs were confirmed by DNA sequencing.

The nucleic acid sequence encoding the CD19 CAR (sense strand) is as follows:

(SEQ ID NO: 9)
gccaccatggctctgcccgtgaccgcactcctcctgccactggctctgct
gcttcacgccgctcgcccacaagtccagcttcaagaatcagggcctggtc
tggtgaagccatctgagactctgtccctcacttgcaccgtgagcggagtg
tccctcccagactacggagtgagctggattagacagcctcccggaaaggg
actggagtggatcggagtgatttggggtagcgaaaccacttactataact
cttccctgaagtcacgggtcaccatttcaaaggataactcaaagaatcaa
gtgagcctcaagctctcatcagtcaccgccgctgacaccgccgtgtatta
ctgtgccaagcattactactatggagggtcctacgccatggactactggg
gccagggaactctggtcactgtgtcatctggtggaggaggtagcggagga
ggcgggagcggtggaggtggctccggaggtggcggaagcgaaatcgtgat
gacccagagccctgcaaccctgtccctttctcccggggaacgggctaccc
tttcttgtcgggcatcacaagatatctcaaaatacctcaattggtatcaa
cagaagccgggacaggcccctaggcttcttatctaccacacctctcgcct
gcatagcgggattcccgcacgctttagcgggtctggaagcgggaccgact
acactctgaccatctcatctctccagcccgaggacttcgccgtctacttc
tgccagcagggtaacaccctgccgtacaccttcggccagggcaccaagct
tgagatcaaaaccactactcccgctccaaggccacccacccctgccccga
ccatcgcctctcagccgctttccctgcgtccggaggcatgtagacccgca
gctggtggggccgtgcatacccggggtcttgacttcgcctgcgatatcta
catttgggcccctctggctggtacttgcggggtcctgctgctttcactcg
tgatcactctttactgtaagcgcggtcggaagaagctgctgtacatcttt
aagcaacccttcatgaggcctgtgcagactactcaagaggaggacggctg
ttcatgccggttcccagaggaggaggaaggcggctgcgaactgcgcgtga
aattcagccgcagcgcagatgctccagcctacaagcaggggcagaaccag
ctctacaacgaactcaatcttggtcggagagaggagtacgacgtgctgga
caagcggagaggacgggacccagaaatggggggaagccgcgcagaaagaa
tccccaagagggcctgtacaacgagctccaaaaggataagatggcagaag
cctatagcgagattggtatgaaaggggaacgcagaagaggcaaaggccac
gacggactgtaccagggactcagcaccgccaccaaggacacctatgacgc
tcttcacatgcaggccctgccgcctcgg.

Preparation of retroviral particles for transduction was carried out as described in Example 8. The detection and quantification of the positive (construct containing) cells was done via FACS analysis, using CD19-Fc and PE anti Fc antibody, and confirmed to express the CD19 CAR gene.

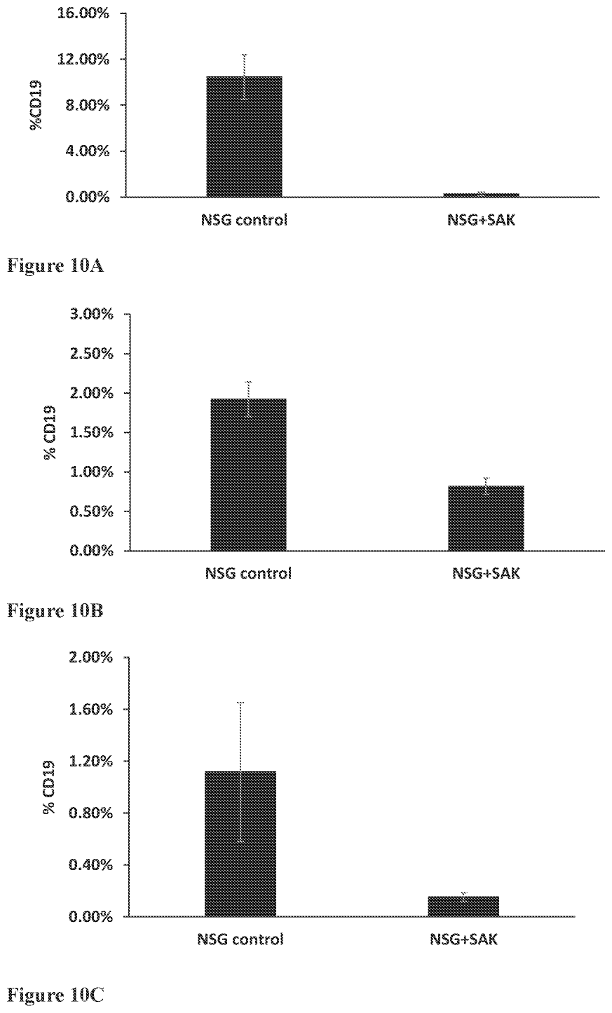

The cytotoxic activity of CD19 CAR SAK cells compared with control SAK cells against CD19 positive cells (Toledo) and CD19 negative cells (U937) was measured. CFSE-based cytotoxicity assays were performed essentially as described in Example 2, using SAK, and CD19 CAR SAK cells as effector cells, and Toledo and U937 cells as the target cells. The results are shown in FIGS. 11A and 11B for CD19⁺ (Toledo) and CD19− (U937) cells, respectively, in which means±standard deviation from triplicate at each ratio are presented.

As can be seen in FIG. 11A, CD19 CAR SAK cells were found to be significantly more effective in eradicating Toledo cells (CD19 positive cells) than the control SAK cells.

CD19 CAR SAK cells exhibited a superior cytotoxic activity at each of the tested E:T ratios. For example, at an E:T ratio of 1:1, CD19 CAR SAK cells exhibited 93% efficiency while control SAK cells at the same ratio showed efficiency of only 54%. In contrast, the cytotoxic activity of CD19 CAR SAK and control SAK against U937 cells was identical, meaning that the enhanced activity of the CD19 CAR cells is specific to the CD19-expressing tumor cells.

These results indicate that CAR SAK could be a valid alternative to conventional CAR T cells, particularly against solid tumors where conventional CAR T cells present a poor therapeutic efficacy.

### Example 10. In Vivo Alloreactivity

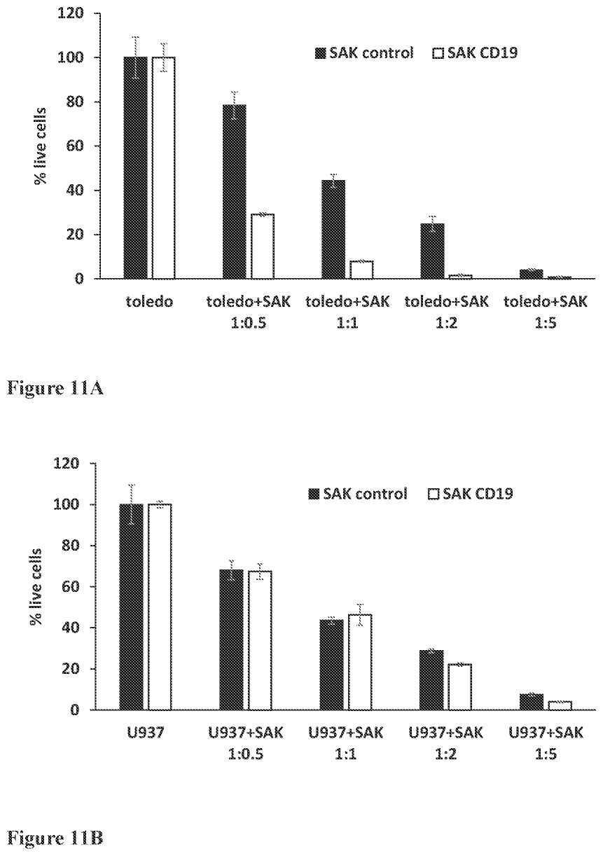

In order to characterize the potential of SAK cells to elicit a graft-versus-host (GVH) reaction and graft versus host disease (GVHD), NSG mice were injected with either SAK cells or PBMC and monitored for a month for GVHD development. To this end, NSG mice were first irradiated with 300 cGy. 24 hrs later, SAK cells or PBMC (purified from human blood) were intravenously (iv) injected (5×10⁶ cells per mouse) via the dorsal tail vein at a final volume of 200 μl. Acute GVHD clinical score was calculated three times a week. Each of the four following parameters was scored 0 (if absent) or 2 (if present): posture, activity, fur texture and skin integrity. Mice weight was also measured three times a week. The results are presented in FIGS. 12A-12C.

At approximately 15 days post injection mice which have received PBMC began showing clinical signs of GVHD (FIG. 12C). Mice appeared scruffy due to lack of grooming. They also began to lose weight. All mice treated with PBMC developed severe acute GVHD and died within 17 days (FIG. 12A). In contrast, mice treated with SAK cells showed minimal signs of GVHD and all animals survived. These mice exhibited mild weight loss compared with PBMC group (FIG. 12B).

These results indicate the compatibility of SAK cells with allogenic use in the clinic, including in the treatment of non-histocompatible subjects, and that SAK cells may be an effective improvement to existing adoptive cell transfer therapeutic protocols.

### Example 11. Evaluation of Cytotoxicity of Cryopreserved SAK Cells

In order to use SAK cells as commercial therapy products, their production and freezing process should advantageously be evaluated before their clinical use. To assess the cytotoxic activity of thawed SAK cells, they were collected 24 h post freezing and co-cultured with MV411 and THP-1 leukemia target cells at different ratios.

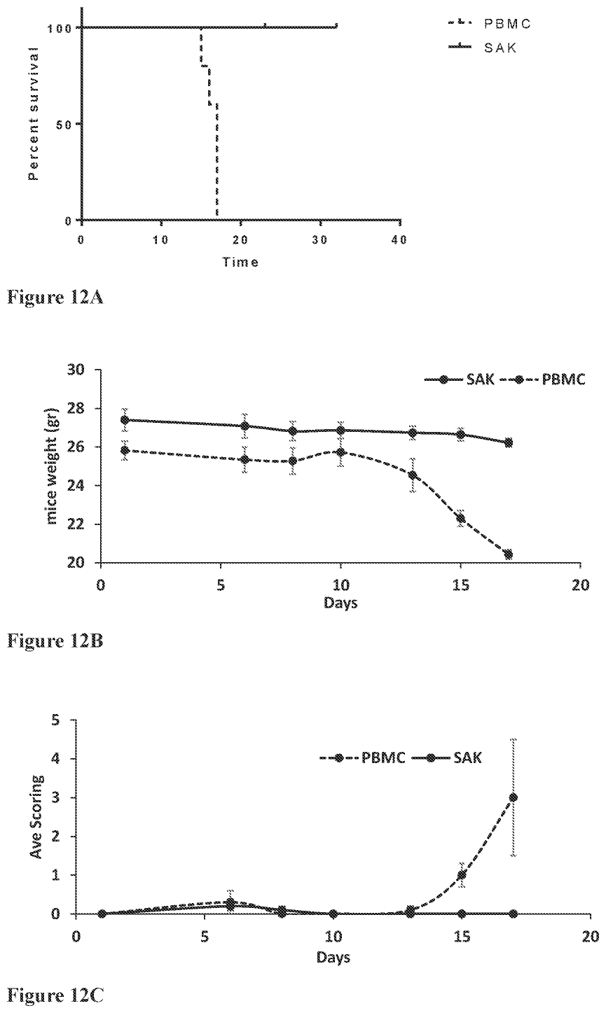

To this end. fresh SAK cells at day 11 of expansion (as described in Example 1) were frozen in freezing medium (containing 90% Fetal Calf Serum (FCS)+10% Dimethyl sulfoxide (DMSO)) and transferred to liquid nitrogen for cryopreservation. The resulting cryopreserved SAK cells were used in cytotoxic assay 24 h after thawing and compared with the cytotoxic activity of fresh SAK cells (which had not undergone freezing and subsequent thawing) at day 11. The results are shown in FIGS. 13A-13B.

As can be seen in FIGS. 13A and 13B, thawed SAK exhibited reduced cytotoxic activity compared with fresh SAK, indicating that the freeze-thaw cycle may have affected the viability and/or the activity of SAK cells to some extent. Nevertheless, cryopreserved SAK cells showed significant and potent cytotoxic activity against MV411 (FIG. 13B) and THP-1 (FIG. 13A) cells even at low ratios of E:T, retaining at least about 70-80% of the cytotoxic activity of the corresponding fresh SAK cells.

Thus, the results indicate that SAK cells have the potential to be used not only close to their time of preparation, but also following cryopreservation. Further, the cell compositions are demonstrated herein to exhibit highly potent cytotoxicity shortly following thawing, without requiring additional steps of expansion or manipulation. Accordingly, cell compositions comprising SAK cells may be manufactured and distributed as a stable product with a long shelf life, facilitated by cryopreservation. This is in contradistinction from certain cell therapies based on other immune cell populations such as NK cells, which have been reported to be more sensitive to cryopreservation, an attribute suggested to be linked to their considerable failure in clinical treatment of solid tumors (Mark et al., Nat Commun 11, 5224 (2020).

### Example 12. Expression of Immune Checkpoint Molecules on SAK Cells

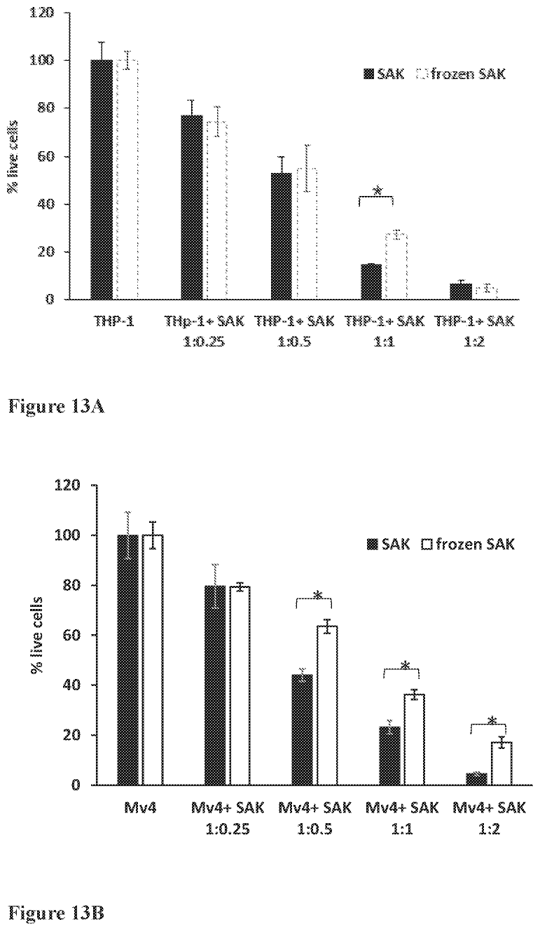

The presence of immune check point molecules on CD8⁺ SAK cells was evaluated as follows. SAK cells, prepared as described in Example, 1 were harvested at day 11 of expansion, washed, stained with either PE-labelled anti Tim-3 antibody (FIG. 14 Top panel, left), APC-labelled anti-CTLA-4 antibody (FIG. 14 Top panel, right), APC-labelled anti-PD-1 antibody (FIG. 14 Middle panel, left), APC-labelled anti-TIGIT antibody (FIG. 14 Middle panel, right) or PE-labelled anti Lag-3 antibody (FIG. 14 Bottom panel, left), at 4° C. for 30 min, and analyzed by flow cytometry. APC or PE-labelled anti-CD8 monoclonal antibodies (Biolegend) and isotype control antibodies were also used. In addition to the inhibitory checkpoint molecules Tim-3, CTLA-4, PD-1, TIGIT and Lag-3, the presence of the activating receptor DNAM-1 (FIG. 14 Bottom panel, right) was also evaluated on SAK cells.

As can be seen in FIG. 14, remarkably, substantially all of the CD8⁺ cells were found to express the activating receptor DNAM-1. In contrast, the inhibitory immune check point molecules were either non-detectable, or could be detected at lower levels. In particular, SAK cells did not express CTLA-4 at all, and expressed low levels of PD-1 and TIGIT, indicating a low potential for inhibition by tumors expressing their corresponding ligands.

When quantifying the proportions of cells expressing the various surface markers in samples of different donors, 99%-100% of the CD8⁺ cells were found to express DNAM-1, about 0% of the cells were found to express CTLA-4, 12%-15% of the cells were found to express PD-1, and 20%-22% of the cells were found to express TIGIT. Surface expression of Lag-3 and Tim-3 was found on about 51%-63% and 54%-97% of the CD8⁺ cells, respectively.

Next, the expression of checkpoint molecules on SAK cells was compared to their expression on CIK cells. The compositions were prepared from the same donor essentially as described in Example 1, and the cells were harvested at day 11 and subjected to phenotypic analysis by flow cytometry, as described above. The results are presented in Table 9 below, in which the mean fluorescent intensity (MFI) measured upon staining for each of the indicated markers in each cell type are shown, as well as the relative expression in SAK cells compared to CIK cells (fold increase, manifested as MFI in SAK cells/MFI in CIK cells, “SAK/CIK”).

TABLE 9
Phenotypic characterization of SAK and CIK cells

| | SAK cells | CIK cells | SAK/CIK |
|---|---|---|---|
| Surface molecule | (MFI) | (MFI) | (fold) |
| DNAM-1 | 14991 | 12461 | 1.20 |
| NKG2D | 4564 | 4236 | 1.08 |
| Lag-3 | 7140 | 3519 | 2.03 |
| Tim-3 | 17517 | 9895 | 1.77 |

As can be seen in Table 9, cells prepared by the different expansion protocols exhibited distinct phenotypes associated with unique expression profiles of surface markers. In particular, SAK cells exhibit markedly higher levels of the tested markers than counterpart CIK cells harvested at day 11. Thus, the results demonstrate that SAK cells may be readily identified and distinguished from other cell compositions by readily available methods. In addition, the presence of surface markers known to be elevated during lymphocyte activation at higher levels on SAK cells than on day-11 CIK cells, further suggests that the SAK cells adapt a “super-activated” phenotype during the improved expansion process.

It is known that NK and T cell functions are regulated and maintained by the balance of inhibitory receptors (e.g. PD-1, TIGIT, Lag-3, Tim-3 and/or CTLA-4) and activating receptors (e.g. DNAM-1). In addition, the blockade of inhibitory immune checkpoint receptors may be crucial in effectively reversing T cell exhaustion and the restoration of the antitumor capacity of T cells. Hence, there is growing interest in the effects of blocking inhibitory immune check point molecules may have on SAK cell activity as well.

It is noted, that SAK cells were demonstrated herein to exhibit a remarkably improved cytotoxic capacity against tumor cells compared to CIK cells after 11 days of expansion (e.g. Examples 3). Without wishing to be bound by a specific theory or mechanism of action, the “super-activated” phenotype of SAK cells, characterized by a unique balance between various receptors and effector molecules as disclosed herein, may be associated with a favorable functional capacity against tumor cells.

In summary, SAK cells produced by the advantageous protocols as disclosed herein were found to display an advantageous profile of cell-surface receptors, including immune checkpoint molecules and chemokine receptors. In particular, SAK cells were found to be characterized by expression of DNAM-1 and CXCR6, known to have a positive effect on the survival, expansion and/or activity of effector immune. Without wishing to be bound by a specific theory or mechanism of action, the results are consistent with the ability to generate ACT compositions characterized by an activated phenotype, capable of minimizing tumor evasion.

### Example 13—Major Histocompatibility Complex Class I (MHC I) Involvement

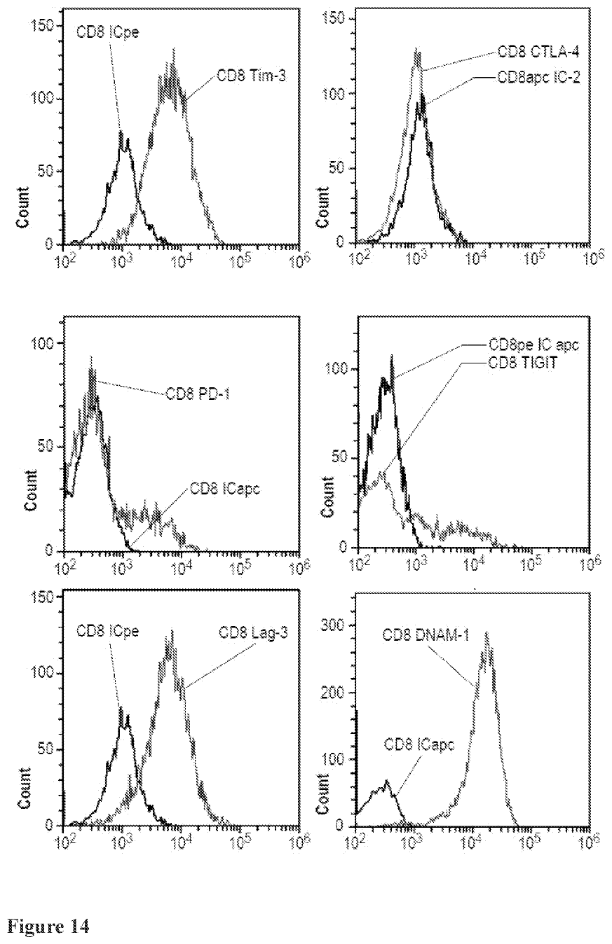

The killing mechanism of SAK cells against MV4-11 leukemic cells (human cell line of biphenotypic B myelomonocytic leukemia, also referred to herein as MV411) was examined using inhibitors of MHC class I. The cytotoxicity assay was performed essentially as described in Example 2, wherein the MV411 cells were incubated with 1 μg/ml of an anti-MHC I antibody (W6/32 clone, Biolegend), one hour before the addition of SAK cells. The results are presented in FIG. 15, in which the degree of tumor killing is shown as the percentile of live cells, calculated as follows: (number of CFSE⁺PI⁻ cells in the presence of effector cells/number of CFSE⁺PI⁻ cells without effector cells)×100. Data represent mean±standard deviation from triplicates at each ratio.

As can be seen in FIG. 15, blocking of MHC class I on MV411 cells inhibited their killing by SAK cells in a dose-dependent manner. However, the inhibition was not complete, and the SAK cells retained at least 40% of their cytotoxic activity even in the presence of the MHC I inhibitor.

Accordingly, the results demonstrate that SAK cells exhibit both MHC I-dependent and MHC I-independent cytotoxic activity against leukemic cells. Notably, both activities did not require HLA allele compatibility, as the effector SAK cells were non-histocompatible. Thus, compositions comprising SAK cells in accordance with the invention may advantageously be used even in the treatment of immune-evading or treatment-refractory tumors, characterized by downregulated MHC-I expression and/or activity.

In summary, the results presented herein demonstrate the suitability of SAK cells for treatment of tumors of various origins in both histocompatible and non-histocompatible subjects. As demonstrated herein, SAK cells exhibited remarkable anti-tumor activity against hematopoietic tumors and solid tumors, retained anti-tumor potency even in the absence of HLA compatibility, and exhibited improved safety, manifested as a markedly reduced risk of invoking graft-versus-host disease (GVHD). Without wishing to be bound by a specific theory or mechanism of action, the cytotoxic activity of SAK cells against various tumor cells may involve NKG2D and/or T cell receptor (TCR)-mediated activities, as exemplified herein.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An adoptive cell transfer (ACT) cell composition, comprising in vitro-expanded peripheral blood mononuclear cells (PBMC), of which 70-85% are CD3⁺CD8⁺ cells expressing NKG2D and granzyme B, at least 70% are CD56⁻ cells, and up to 5% are CD3⁻ cells, said composition exhibiting significant cytotoxic activity against tumor cells in vitro and in vivo.

2. The cell composition of claim 1, wherein 7-12% of the cells are CD3⁺CD4⁺ cells, 20-28% are CD3⁺CD56⁺ cells, and 10-14% are CD3⁺CD8⁺CD56⁺ cells.

3. The cell composition of claim 2, wherein the amount of CD3⁺CD8⁺ cells in the composition is 6-8 times the amount of the CD3⁺CD4⁺ cells in the composition.

4. The cell composition of claim 1, comprising less than 3% CD3⁻CD56⁺ cells.

5. The cell composition of claim 1, wherein at least 40% of the CD3⁺CD8⁺ cells are characterized by surface expression of at least one marker selected from the group consisting of perforin, Fas-ligand (FasL), CXCR4 and CXCR2.

6. The cell composition of claim 1, wherein said CD3⁺CD8⁺ cells are further characterized as DNAM-1⁺, CTLA-4⁻ cells.

7. The cell composition of claim 1, wherein at least 90% of said CD3⁺CD8⁺ cells are characterized by surface expression of CXCR3 and at least 30% of said CD3⁺CD8⁺ cells are characterized by surface expression of CXCR6.

8-11. (canceled)

12. The cell composition of claim 1, capable of specifically eradicating hematopoietic tumor cells in vitro at a ratio of 0.25:1 within 24 hours.

13-15. (canceled)

16. The cell composition of claim 1, prepared by a process comprising: expanding PBMC in the presence of effective amounts of IL-2 and a CD3 activator for at least nine days, wherein effective amounts of the IL-2 and CD3 activator are supplemented every 48-96 hours.

17. The cell composition of claim 16, wherein the incubation is performed in the absence of other cytokines or antibodies.

18. The cell composition of claim 1, which cells are genetically modified.

19. The cell composition of claim 18, wherein the cells are genetically modified to expresses at least one of a chimeric antigen receptor (CAR), a cytokine, a chemokine, a cytokine or chemokine receptor, or any combination thereof.

20-21. (canceled)

22. The cell composition of claim 18, which cells are genetically modified to express a modified CXCR4 receptor comprising a mutation or truncation at the C-terminal tail domain.

23. The cell composition of claim 18, which expresses any one of exogenous interleukin-2 (IL-2), exogenous IL-15, exogenous IL-21, or any combination thereof.

24. (canceled)

25. A process for producing a cell composition for adoptive cell transfer (ACT), comprising:
provided a peripheral blood mononuclear cells (PBMC) sample,
expanding the PBMC in the constant presence of effective amounts of IL-2 and a CD3 activator for at least nine days, and in the absence of other cytokines or antibodies, wherein effective amounts of the IL-2 and CD3 activator are supplemented every 48-96 hours,
wherein the number of viable cells in the culture is enhanced by at least 20-fold, and a cell composition comprising at least 5×10⁶-10×10⁸ viable cells is obtained.

26. The process of claim 25, wherein expansion is performed in the constant presence of said IL-2 and CD3 activator as sole exogenously-added cell stimulators.

27. The process of claim 25, further comprising expanding the cells in the presence of 500-1500 IU/ml IL-2 and 10-60 ng/ml anti-CD3 antibody, which are re-supplemented every 48-96 hours, for between 10-16 days.

28-31. (canceled)

32. The process of claim 25, further comprising genetically modifying the cells.

33. The process of claim 32, comprising genetically modifying said cells to express at least one of a chimeric antigen receptor (CAR), a cytokine, a chemokine, a receptor of a cytokine or chemokine, or any combination thereof.

34-55. (canceled)

56. A method of treating a tumor in a subject in need thereof, comprising contacting the tumor cells with a therapeutically effective amount of the cell composition of claim 1, thereby treating the tumor in the subject.

57-61. (canceled)

62. The method of claim 56, wherein the tumor is a hematopoietic tumor or a solid tumor.

63. The method of claim 62, wherein said tumor is selected from the group consisting of leukemia, multiple myeloma, a prostate tumor or mesothelioma.

64. The method of claim 62, wherein said tumor is characterized by expression of at least one NKG2D ligand.

65. The method of claim 64, wherein said tumor expresses a plurality of NKG2D ligands selected from the group consisting of: MICA, MICB, HLAE, ULBP1, ULBP256, and ULBP3.

66-68. (canceled)

69. The method of claim 56 wherein said tumor is resistant to treatment by at least one checkpoint molecule inhibitor.

70-73. (canceled)


SEQUENCE LISTING (9 sequences)

SEQ ID NO: 1 - DNA, 1020 bp, Artificial Sequence
  anti-human CD123 CAR
atggccctgcctgtgacagccctgctgctgcctctggctctgctgctgcatgccgctagacccgatattgtcctcactcaatcgccggactcactggcggtgtccctcggagagagggcgacgatcaattgccgggcttccgaatccgtcgataactacggaaacacctttatgcactggtaccaacagaagccaggacagccaccaaagctgttgatctaccgcgcttcaaaccttgagtcgggtgtgccggaccgcttcagcggcagcggttccagaaccgactttaccctcaccatcagctcgctgcaggccgaagatgtcgccgtctattactgccaacagagcaacgaagatccgcctactttcggacaggggactaaactggaaatcaagggcggaggaggctcgggtggaggaggatcgggaggaggcgggtccggtggtggcggatcgcaaatccagctggtgcagtccggcgcagaagtgaagaagccgggagcgtccgtgaaagtgagctgcaaggcctcagggtacatcttcaccaattacggcatgaattgggtgcggcaggcacccggacagcgcctggagtggatgggctggatcaacacttacaccggggaaagcacgtactcggccgacttcaaaggacgggtgaccattaccctggatacctcggcctcaaccgcttacatggagctctcatcacttagatccgaggacactgccgtctactactgtgcaaggagcggaggctacgaccctatggactattggggacaaggcactactgtgactgtgtcgtccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctaa

SEQ ID NO: 2 - DNA, 661 bp, Artificial Sequence
  IRES nucleic acid sequence
gaattcccgggtcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgc

SEQ ID NO: 3 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 4 - DNA, 486 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 5 - DNA, 54 bp, Artificial Sequence
  2A element
gagggcaggggaagtctactaacatgcggggacgtggaggaaaatcccggccca

SEQ ID NO: 6 - DNA, 720 bp, Artificial Sequence
  GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 7 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 8 - DNA, 1013 bp, Homo sapiens
atgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtgatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagtaa

SEQ ID NO: 9 - DNA, 1479 bp, Artificial Sequence
  CD19 CAR
gccaccatggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg